SEQUENCE LISTING (15 sequences)

SEQ ID NO: 1 - protein, 18 aa, Artificial Sequence
  Description of Artificial Sequence Synthetic peptide
CAALPKINRSASEPSLHR

SEQ ID NO: 2 - protein, 248 aa, Geodia cydonium
MSEAPEPSPLTTKARIAEQAERYEDMAKFMKEVTEKATEELSPDDRNLLSVAYKNVVGTRRSSWRVISSIEQKSSDENKKEMAKAYREKIEKELDEICNEVLTLLDDHLLKKATSDESKVFYQKMKGDYYRYLAEVTPSGDKREDVVVKAKDSYNKALEVATEKMPPTHPIRLGLALNFSVFHYEIQNAPEKACEMAKKAFDEAIAELDNLKEDSYKDSTLIMQLLRDNLTLWTSEGQTEEQEDGGDN

SEQ ID NO: 3 - protein, 247 aa, Rattus norvegicus
MVDREQLVQKARLAEQAERYDDMAAAMKNVTELNEPLSNEERNLLSVAYKNVVGARRSSWRVISSIEQKTSADGNEKKIEMVRAYREKIEKELEAVCQDVLSLLDNYLIKNCSETQYESKVFYLKMKGDYYRYLAEVATGEKRATVVESSEKAYSEAHEISKEHMQPTHPIRLGLALNYSVFYYEIQNAPEQACHLAKTAFDDAIAELDTLNEDSYKDSTLIMQLLRDNLTLWTSDQQDDDGGEGNN

SEQ ID NO: 4 - protein, 246 aa, Homo sapiens
MGDREQLLQRARLAEQAERYDDMASAMKAVTELNEPLSNEDRNLLSVAYKNVVGARRSSWRVISSIEQKTMADGNEKKLEKVKAYREKIEKELETVCNDVLSLLDKFLIKNCNDFQYESKVFYLKMKGDYYRYLAEVASGEKKNSVVEASEAAYKEAFEISKEQMQPTHPIRLGLALNFSVFYYEIQNAPEQACLLAKQAFDDAIAELDTLNEDSYKDSTLIMQLLRDNLTLWTSDQQDEEAGEGN

SEQ ID NO: 5 - protein, 245 aa, Ovis sp.
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNRSQPESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTQGDEAEAGEGGEN

SEQ ID NO: 6 - protein, 235 aa, Xenopus laevis
AKLSEQAERYDDMAASMKAVTELGAELSNEERNLLSVAYKNVVGARRSSWRVISSIEQKTEGDNKRQQMAREYREKVETELQDICKDVLDLLDRFLVPNATPPESKVFYLKMKGDYYRYLSEVASGDSKQETVASSQQAYQEAFEISKSEMQPTHPIRLGLALNFSVFYYEILNSPEKACSLAKSAFDEAIRELDTLNEESYKDSTLIMQLLRDNLTLWTSENQGEEADNVEGDN

SEQ ID NO: 7 - protein, 246 aa, Rattus norvegicus
MTMDKSELVQKAKLAEQAERYDDMAAAMKAVTEQGHELSNEERNLLSVAYKNVVGARRSSWRVVSSIEQKTERNEKKQQMGKEYREKIEAELQDICSDVLELLDKYLILNATHAESKVFYLKMKGDYFRYLSEVASGDNKQTTVSNSQQAYQEAFEISKKEMQPTHPIRLGLALNFSVFYYEILNSPEKACSLAKTAFDEAIAELDTLNEESYKDSTLIMQLLRDNLTLWTSENQGDEGDAGEGEN

SEQ ID NO: 8 - protein, 245 aa, Rattus norvegicus
MEKTELIQKAKLAEQAERYDDMATCMKAVTEQGAELSNEERNLLSVAYKNVVGGRRSAWRVISSIEQKTDTSDKKLQLIKDYREKVESELRSICYEVLELLDKYLIANATNPESKVFYLKMKGDYFRYLAEVACGDDRKQTIENSQGAYQEAFDISKKEMQPTHPIRLGLALNFSVFYYEILNNPELACTLAKTAFDEAIAELDTLNEDSYKDSTLIMQLLRDNLTLWTSDSAGEECDAAEGAEN

SEQ ID NO: 9 - protein, 248 aa, Drosophila melanogaster
MSTVDKEELVQKAKLAEQSERYDDMAQAMKSVTETGVELSNEERNLLSVAYKNVVGARRSSWRVISSIEQKTEASARKQQLAREYRERVEKELREICYEVLGLLDKYLIPKASNPESKVFYLKMKGDYFRYLAEVATGDARNTVVDDSQTAYQDAFDISKGKMQPTHPIRLGLALNFSVFYYEILNSPDKACQLAKQAFDDAIAELDTLNEDSYKDSTLIMQLLRDNLTLWTSDTQGDEAEPQEGGDN

SEQ ID NO: 10 - protein, 248 aa, Caenorhabditis elegans
MSDGKEELVNRAKLAEQAERYDDMAASMKKVTELGAELSNEERNLLSVAYKNVVGARRSSWRVISSIEQKTEGSEKKQQMAKEYREKVEKELRDICQDVLNLLDKYLIPKAGAAESKVFYLKMKGDYFRYLAEVASGDDRNSVVEKSQQSYQEAFDIAKDKMQPTHPIRLGLALNFSVFFYEILNAPDKACQLAKQAFDDAIAELDTLNEDSYKDSTLIMQLLRDNLTLWTSDAATDDTDANETEGGN

SEQ ID NO: 11 - protein, 248 aa, Homo sapiens
MERASLIQKAKLAEQAERYEDMAAFMKGAVEKGEELSCEERNLLSVAYKNVVGGQRAAWRVLSSIEQKSNEEGSEEKGPEVREYREKVETELQGVCDTVLGLLDSHLIKEAGDAESRVFYLKMKGDYFRYLAEVATGDDKKRIIDSARSAYQEAMDISKKEMPPTNPIRLGLALNFSVFHYEIANSPEEAISLAKTTFDEAMADLHTLSEDSYKDSTLIMQLLRDNLTLWTADNAGEEGGEAPQEPQS

SEQ ID NO: 12 - protein, 6 aa, Artificial Sequence
  Description of Artificial Sequence Synthetic illustrative binding motif
RSXSXP

SEQ ID NO: 13 - protein, 12 aa, Artificial Sequence
  Description of Artificial Sequence Synthetic illustrative formula motif
CXXSXXSXXSXX

SEQ ID NO: 14 - protein, 8 aa, Artificial Sequence
  Description of Artificial Sequence Synthetic peptide
VAYKNVVG

SEQ ID NO: 15 - DNA, 20 bp, Artificial Sequence
  Description of Artificial Sequence Synthetic degenerate primer
gtkgcctacaaraaygtggt

(19) United States
(12) Patent Application Publication
(10) Pub. No.: US 2005/0009094 A1
(43) Pub. Date: Jan. 13, 2005

(54) Use of 14-3-3 proteins and a method for determining the same in the fluids or tissues of organisms

(76) Inventors: Werner E G Mueller, Wiesbaden (DE); Heinz C Schroeder, Wiesbaden (DE)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/479,925

(22) PCT Filed: Jun. 4, 2002
(86) PCT No.: PCT/EP02/06139

(30) Foreign Application Priority Data
Jun. 6, 2001 (DE) 101 31 912.6

(51) Int. Cl. G01N 33/53

(52) U.S. Cl. 435/7.1; 530/396

(57) ABSTRACT

The object of the present invention is to provide a method for the detection and/or quantification of the 14-3-3 proteins or their isoforms for early stage diagnosis of TSE-diseases, which method allows to perform the diagnosis in the living organism. It is furthermore an object, to detect a contamination of the sample by the parallel determination of a second antigen. This object according to the invention is solved by making use of the biochemical characteristics of the members of the 14-3-3 protein family, which bind to specific amino acid motifs like X(n)-XSXXSXXSX-X(n) or to the motif RSXpSXP (SEQ ID NO: 12) within peptides or proteins. For determining one or more isoforms or the entirety of the 14-3-3 protein(s) and for specific binding, one uses modified solid phases like e.g. microtiter plates, which are coated with a synthetic or natural peptide containing a binding motif for 14-3-3 proteins, e.g. a chemically synthesised peptide having the motif CAALPKINRSApSEPSLHR (SEQ ID NO: 1). After the addition of the extracts or bodily fluids to be investigated the detection and quantification of the generated peptide-14-3-3 protein complexes is accomplished by means of labeled antibodies. The use of the 14-3-3 protein family and/or of individual isoforms of the 14-3-3 proteins according to the invention can be employed as an effect monitor or biomonitor in aquatic invertebrates after environmental effects like the presence of polychlorinated biphenyls (PCBs), (xeno)estrogens etc. Moreover, the method can be used for early diagnosis of TSE-diseases like e.g. Creutzfeldt-Jakob disease (CJD) and its novel form (variant) in young persons (vCJD) and Bovine Spongiform Encephalopathy (BSE) or comparable diseases. Thus a diagnostic marker (surrogate marker) is available, which can be used in the living organism as a screening marker, confirmation marker or single marker.

### BACKGROUND OF THE INVENTION

The present invention relates to the use of one ore more isoforms from the 14-3-3 protein family for the universal, indirect detection of metabolic alterations in cells or complex cell systems. The use of isoforms of the 14-3-3 protein(s) as a biomarker can be employed for the detection of environmental stress both in environmental samples (water and soil samples) and in animals and cells, with this environmental stress being caused by natural and anthropogenic environmental pollutants, particularly by polychlorinated biphenyls (PCBs) and estrogens/xenoestrogens [(xeno)estrogens]. The present invention also relates to the development and employment of a novel method (an ELISA-method, called “14-3-3 Protein-Capture Assay”) for the quick qualitative and quantitative determination of the 14-3-3 proteins. The detection methods can also be employed for detecting the presence of 14-3-3 protein isoforms in bodily fluids from humans and animals being infected with pathogens of prion diseases. This is needed, since for these diseases there is currently no test-kit available for a surrogate marker, which allows to be determined in living organisms. Such a surrogate marker can be employed for early stage analytics and confirmation analytics of an infection with or a generation or an increased concentration of the pathogenic prion protein PrP^{Sc}, which is regarded to be the pathogenic cause of the Transmissible Spongiform Encephalopathies (TSE). Prominent examples of TSE diseases, in which isoforms of the 14-3-3 proteins can be used as surrogate markers, include:

Creutzfeldt-Jakob disease (CJD) and its novel form in young persons (variant CJD, vCJD)

Gerstmann-Sträussler-Scheinker syndrome (GSS)

Fatal familial insomnia (FFI)

Kuru

Scrapie (Traberkrankheit; Gnubberkrankheit; tremblente de mouton)

Bovine Spongiform Encephalopathy (BSE)

Transmissible mink encephalopathy (TME)

Spongiform encephalopathies in wild ruminants

Feline spongiform encephalopathy (FSE)

Chronic Wasting Disease of Cervine Animals (CWD)

At present, there exists no such surrogate marker, which would make it possible to obtain early information or perform early stage diagnostics for CJD and vCJD in patients, for BSE in living cattle, for Scrapie in the living sheep or for other TSE diseases in other animals. By employing specific antibodies directed against different isoforms of 14-3-3 protein, one is also allowed to achieve an improved distinction of prion diseases from other diseases, in which increased concentrations of 14-3-3 proteins in bodily fluids may be present.

### STATE OF THE ART AND CURRENT KNOWLEDGE

The heat shock protein HSP70 was established as a biomarker serving as an indicator of environmental stress, especially in the form of osmotic stress or of heavy metal pollutants (Koziol et al., Canad. Technical Report of Fisheries and Aquatic Sci. 2093, 104-109, 1996; Krasko et al., Aquatic Toxicol. 37, 157-168, 1997; Koziol et al., Marine Ecol., Progr. Ser. 154, 261-268, 1997). HSP70 is a molecular Chaperone, the main function of which is to control and maintain the functionally correct folding of a protein (Becker and Craig, Eur. J. Biochem. 219, 11-23, 1994).

In contrast to this, the 14-3-3 protein (Aitken et al., Trends Biochem. Sci 17, 498-501, 1992) binds to functional molecules, like (i) to receptors (e.g. the Adrenodoxin precursor) (Alam et al., J. Biochem. 116, 416-425, 1994), (ii) to signal transduction proteins like Raf-1 (Muslin et al., Cell 84, 889-897, 1996) or (iii) to key molecules of apoptosis (like the BAD molecule or A20) (Zha et al., Cell 87, 619-628, 1996) and prevents these molecules from being transported to their target sites.

The isoforms of the 14-3-3 proteins thus constitute novel chaperones, the function of which is to bind to other proteins and to “fix” them in specific cellular compartments. The 14-3-3 proteins control essential functions of the cell and “immobilise” its metabolism (Aitken et al., Trends Biochem. Sci. 17, 498-501, 1992). First being discovered as neuronal proteins, the 14-3-3 proteins nowadays are regarded as ubiquitous proteins, which are of crucial relevance for the mentioned processes; the family of 14-3-3 proteins comprises at least seven isoforms (Aitken et al., Trends Biochem. Sci. 17, 498-501, 1992).

A summary of data for 14-3-3 proteins:

Family of 30 kDa proteins

Proteins with a high frequency in the nervous system

Important function in signal transduction, in cell cycle regulation, in the regulation of apoptosis and the cellular stress response

Homo- or heterodimers

They interact with a variety of ligands like PKC, Raf-1, MEK kinases, Cbl and Bad

Those proteins, which interact with the 14-3-3 proteins, are in possession of a phosphorylated 14-3-3 protein binding motif, RSXpSXP

At least 7 isoforms of the 14-3-3 proteins are present in mammalian cells

There exist known methods for detecting one or more isoforms from the 14-3-3 protein family by means of immunoblotting (Western Blot) methods or by ELISA methods. These immunoblotting methods exclusively employ antibodies as 14-3-3 protein detecting molecules, whereat the entirety of 14-3-3 proteins or individual 14-3-3 protein isoforms are determined. Starting from this method, different research groups use varying detection methods for protein detection like enzymatic or fluorescence detection.

The patent application WO 99/46401 employs a binding peptide for the determination of the 14-3-3 proteins. However, only a chemically synthesized peptide having the sequence CAALPKINRSApSEPSLHR (pS=phosphoserine) is employed. This only allows for a restricted use of this test variant in different species.

Immunoblotting (Western Blot) detection methods for 14-3-3 proteins have been described in the patents WO 97/38315 and WO 97/33601, which techniques however have known and described disadvantages. In the following, these problems in the immunoblot (Western Blot) detection of 14-3-3 proteins are shortly summarised:

No detection of low concentrations of the protein

No detection of slight changes in the liquor concentration of 14-3-3 protein in the disease progression of the novel Creutzfeldt-Jakob disease (vCJD) or in iatrogenic CJD

### ADVANTAGES OF THE INVENTION

The inventors have shown, that a novel “guiding” chaperone, 14-3-3 protein(s), is expressed in lower aquatic invertebrates after the impact of polychlorinated biphenyls (PCBs). Thus, a new biomarker for PCB—at least for aquatic invertebrates—has been found; the present methods for the detection of PCBs in invertebrates are insufficient. Moreover is has been found, that the expression of 14-3-3 protein(s) is also inducible by (xeno)estrogens. The biomarker 14-3-3 protein(s) was/were employed for the purpose of an effector monitoring of PCB and of (xeno)estrogens as a bioindicator, thereby employing the fresh water mussel Corbicula fluminea, the North Sea dab Limanda limanda and the Mediterranean Sponge Suberites domuncula.

Moreover, it has been shown, that the detection and quantification of the 14-3-3 proteins or at least of one isoform from this protein family can allow for an early stage detection of TSE diseases like Creutzfeldt-Jakob disease (CJD) and its novel form in young persons (vCJD), Gerstmann-Sträussler-Scheinker syndrome (GSS), Fatal familial insomnia (FFI), Kuru, Scrapie (Traberkrankheit; Gnubberkrankheit; tremblente de mouton), Bovine Spongiform Encephalopathy (BSE), Transmissible mink encephalopathy (TME), Chronic Wasting Disease of cervine animals (CWD), Spongiform encephalopathies in wild ruminants and Feline spongiform encephalopathy (FSE). The literature describes detection methods determining and/or quantifying the entirety of the 14-3-3 proteins. The determination of the total concentration however cannot reflect a disease progression or a change in metabolism.

A biomarker of such kind (surrogate marker), which is able to early detect environmental effects or TSE diseases, like e.g. BSE in the living animal, is not known.

### OBJECT OF THE INVENTION

It is thus an object of the invention described in this patent document to avoid or to reduce the disadvantages of the currently used methods. A further object is to detect a contamination of the sample by a parallel determination of a second antigen.

It is also an object of the invention to establish a detection method and a quantification method for the 14-3-3 proteins or their subforms to allow for early stage differential diagnostics of TSE diseases like Creutzfeldt-Jakob disease (CJD) and its novel form in young persons (vCJD), Gerstmann-Sträussler-Scheinker syndrome (GSS), Fatal familial insomnia (FFI), Kuru, Scrapie (Traberkrankheit; Gnubberkrankheit; tremblente de mouton), Bovine Spongiform Encephalopathy (BSE), Transmissible mink encephalopathy (TME), Chronic Wasting Disease of cervine animals (CWD), Spongiform encephalopathies in wild ruminants and Feline spongiform encephalopathy (FSE). This is intended also to be employed for the analytics of disease progression (in the living organism) and for confirmation analytics of TSE diseases. In particular the confirmation analytics is essential with regard to aspects of quality and safety und is at present not available, because in the afore mentioned diseases only a symptomatic determination or a determination of the PrP^{Sc} protein (“Scrapie prion protein”, pathogenic form of the prion protein) is available. Another object of the invention is to further decrease the yet insufficient detection limit (1-10 ng/ml) of the method described in the patent application WO 99/46401 in order to allow for the performance of trustworthy determination also in the lower range of 14-3-3 protein in human and animals. Moreover, a quality control of the determination is made possible by introducing a second marker/indicator assay.

### DESCRIPTION OF THE INVENTION

The present invention relates to the use of one or more isoforms from the 14-3-3 protein family for a universal detection of alterations of metabolism in cells or complex cellular systems as well as in tissues and organic fluids. The methods described in the following (“14-3-3 Protein-Capture Assay”) can also be employed for the detection of 14-3-3 proteins in the bodily fluids of humans and animals being infected with the pathogen of prion diseases. The object is achieved according to the invention by making use of the biochemical properties of the 14-3-3 proteins, which bind to specific amino acid motifs like X(n)-XSXXSXXSX-X(n) (with X=variable amino acid and S=serine or phosphoserine) or to the motif RSXpSXP within peptides or proteins.

The method according to the invention relates to the detection, determination and/or the quantification of an isoform and/or the entirety of the 14-3-3 protein isoforms from the 14-3-3 protein family in the living or dead organism in the human and in the veterinary field, which is characterised in that in a biological sample at least one isoform and/or the entirety of the 14-3-3 protein isoforms from the 14-3-3 protein family is/are contacted with synthetic or natural peptides comprising an amino acid sequence motif (or motifs) of the type X(n)-XSXXSXXSX-X(n), in particular CX(n)-XSXXSXXSX-X(n), wherein X is a variable amino acid and S is serine or phosphoserine, and/or RSXpSXP and/or one or more antibodies directed against purified or recombinant 14-3-3 proteins or 14-3-3 protein isoforms or the peptides derived thereof, and that the 14-3-3 protein isoform and/or the entirety of the 14-3-3 protein isoforms is detected, determined and/or quantified by means of affinity binding.

Preferably, a method according to the invention is characterised in that a solid phase being coated with the synthetic or natural peptides or antibodies is used for the determination and specific binding or concentration of the 14-3-3 proteins. Moreover, it is preferred that a microtiter plate is used as the solid phase.

According to a further embodiment, the synthetic or natural peptides used for the specific binding or concentration of the 14-3-3 proteins are bound by means of maleimide activated microtiter plates by the reaction of the sulfhydryl group at the N-terminal cysteine. After having added the extracts or bodily fluids to be analysed to the wells of the microtiter plates or to another solid phase, it is moreover possible to detect and quantify the generated peptide-14-3-3 protein complexes by means of radionucleotide-, dye- or enzyme-labeled antibodies or to use a streptavidin coated microtiter plate or solid phase, to which a biotinylated peptide with the above mentioned binding motif of the 14-3-3 protein is coupled. In order to generate the peptide-14-3-3 protein complexes, the incubation of a biotinylated peptide with the sample is performed either before or after the coupling of the peptide to the plate.

According to another preferred method according to the present invention, a carbodiimide or epoxide activated microtiter plate or solid phase is employed, to which a peptide having the above mentioned binding motif/motifs for the 14-3-3 proteins binds, whereat—for the generation of the peptide-14-3-3 protein complexes—the incubation of the sample with the peptide is performed either before or after the peptide has bound to the plate.

Another preferred method according to the present invention is characterised in that the determination of 14-3-3 protein is performed with a solid phase and a mobile phase, whereat the binding peptide and/or the above mentioned antibody is coupled to the solid phase and the 14-3-3 protein to be determined is present in the mobile phase.

In a particularly preferred version, the method of the present invention is performed in the form of a so-called “sandwich assay”. Thereby, at least one isoform from the 14-3-3 protein family and/or the entirety of this family is isolated by means of one or more specific capturing antibodies, which is/are bound covalently or in an alternative manner to solid phases, in particular to microtiter plates, followed by an immediate or delayed determination or quantification by means of a second, specific detection antibody. The sandwich assay thus is present in the form of an antibody-14-3-3 protein(s)-antibody construct. Preferably, the determination is accomplished in the form of a competitive assay.

Here, a competition takes place e.g. between the 14-3-3 protein to be determined in the sample and purified or recombinant 14-3-3 proteins or 14-3-3 protein isoforms or peptide fragments thereof, all of them competing for the same binding site at the immobilised binding peptide or antibody at the solid phase. Alternatively, it may be a competition between soluble 14-3-3 protein binding peptides in the mobile phase and the immobilised 14-3-3 protein binding peptide at the solid phase, both competing for the 14-3-3 protein in the sample.

According to another preferred method of the present invention, the method is combined in the same assay system or in the same kit with the determination of another biomarker (surrogate marker) or of a pathogenic agent being present in the same sample mixture by its binding to the same or to another solid phase in the form of a combination assay.

Also preferred is a method according to the present invention in the form of a capture assay, comprising the combination of binding the molecule marker to be determined to (a) a peptide recognition sequence and (b) to an antibody.

According to a further aspect of the present invention, the method according to the invention combines the determination of 14-3-3 protein with the determination of a quality marker for the sample to be analysed.

The biological sample to be used for determination/identification or detection may comprise cells, cell associations, tissues, organic fluids or bodily fluids like blood, serum, plasma, liquor, lacrimal secretion, milk secretion or urine. In general, all 14-3-3 protein containing samples are suitable, whereat these samples may also be prepared or pre-treated in suitable manner, e.g. by filtration, column matrices, chromatography, precipitation or the like.

According to a further aspect of the present invention, the method is used for the determination of 14-3-3 binding proteins or 14-3-3 specific antibodies, whereat one measures the decrease of the binding of a pre-selected amount of 14-3-3 protein to the solid phase in the presence of the above mentioned marker molecules to be determined.

According to a further aspect of the present invention, the method for detecting and quantifying the 14-3-3 proteins or their isoforms is used for the diagnosis of TSE diseases like Creutzfeldt-Jakob disease (CJD) and its novel form in young persons (vCJD), Gerstmann-Sträussler-Scheinker syndrome (GSS), Fatal familial insomnia (FFI), Kuru, Scrapie (Traberkrankheit; Gnubberkrankheit; tremblente de mouton), Bovine Spongiform Encephalopathy (BSE), Transmissible mink encephalopathy (TME), Chronic Wasting Disease of cervine animals (CWD), Spongiform encephalopathies in wild ruminants and Feline spongiform encephalopathy (FSE) in living and/or dead organisms, as well as in other diseases associated with changes of the 14-3-3 protein concentration, and as well for progression monitoring of therapies against diseases being associated with the 14-3-3 protein concentration.

According to a further aspect of the present invention, the method is used for early stage diagnostics of BSE or Creutzfeldt-Jakob disease of the old or novel variant in the living or dead patient or organism, thereby employing blood serum, plasma or other (human) bodily fluids.

According to another aspect, the 14-3-3 protein family or at least one isoform from the 14-3-3 protein family can be employed as a biomarker for detecting effects of xenobiotics of all kind or effects of natural environmental poisons on aquatic invertebrates or other organisms including the human.

In the scope of the present invention, one can also employ another solid phase of any kind as an alternative to the microtiter plates. Possible examples for this are resin matrices, beads, membranes or other carrier materials known to the expert.

A “quality marker” in the sense of the present invention is understood as a protein marker or another marker molecule, which indicates either a contamination of the sample with foreign or bodily fluids, cell lysis components, microbes as well as cells and tissues or which indicates an incorrect storage (too high temperatures or storage for a too long time). The quality marker may also be specific for the individual or the species, thus allowing an identification of the sample (detection of an intended or unintended exchange of samples of different individuals or species by the determination of an individual- or species-specific distinctive genetic or immunological marker).

The methods used by the inventors avoid the disadvantages of the current methods. A drawback of the 2D- or 1D-Western Blot methods is their poor detection limit and their accomplishment, which can only be realised by qualified specialists. An ELISA used in the US, which employs two different antibodies on the basis of a polyclonal antibody serum as a so-called Capture-Antibody (Capture-AB) and a monoclonal AB as a detection antibody (Kenney et al., Ann. Neurol. 48: 395-398, 2000) exhibited a multiplicity of unspecific reactions, which is unacceptable for a broad application in routine analytics.

For the determination of the 14-3-3 protein(s) and for its/their specific binding (and concentration) we use solid phases like microtiter plates, which are coated with a peptide containing a binding motif of 14-3-3 proteins, e.g. a chemically synthesized peptide with the motif CX(n)-XSXXSXXSX-X(n) (with X=variable amino acid and S=serine or phosphoserine), e.g. CAALPKINRSApSEPSLHR (pS=phosphoserine). Such peptides can e.g. be coupled by using maleimide activated microtiter plates, to which the 14-3-3 proteins bind with a high affinity, whereat this coupling is based on the reaction of the sulfhydryl group of the N-terminal cysteine. After having added the extracts or bodily fluids to be investigated to the wells of the microtiter plates or other receptacles or solid phases, the detection and quantification of the generated peptide-14-3-3 protein complex is e.g. accomplished by means of radionucleotide-, dye- or enzyme-labeled antibodies.

The invention will now be illustrated in the following by means of the given examples and with reference to the accompanying figures. The examples are merely intended as an illustration and further explanation of the invention. The examples do not restrict the scope of the claims.

What is shown is:

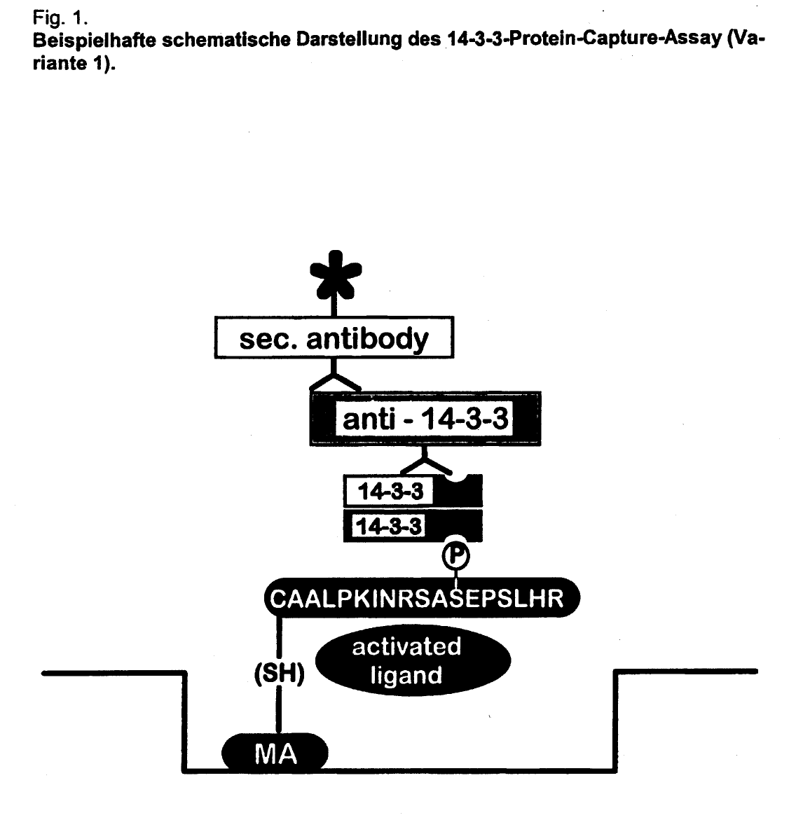

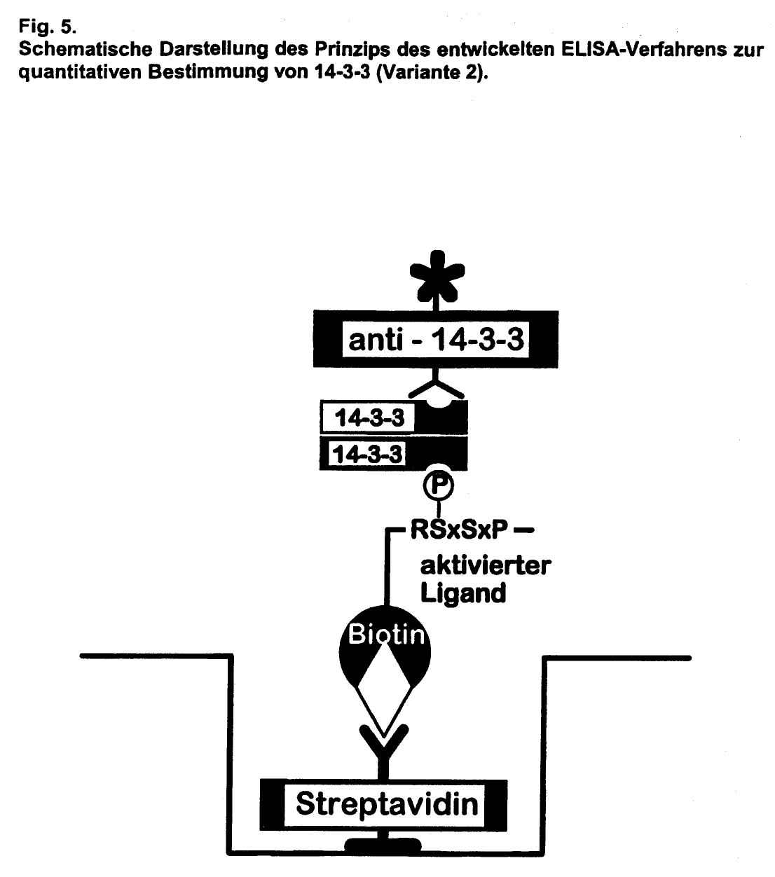

FIG. 1: The schematic depiction of the principle of the developed 14-3-3 protein binding assay (14-3-3 protein capture assay) (variant 1). The 14-3-3 protein dimer binds to an “activated ligand”, a chemically synthesized peptide (pS=phosphoserine), which is covalently bound to a maleimide activated microtiter plate. The generated complexes are then detected by means of an antibody directed against the 14-3-3γ isoform, followed by adding a second antibody, which is coupled to peroxidase or alkaline phosphatase, and substrate. The 14-3-3 protein constitutes a novel chaperone, the function of which is to bind to other proteins and to “fix” them in specific cellular compartments. 14-3-3 controls essential functions of the cell and “immobilises” its metabolism. Firstly discovered as a neuronal protein, 14-3-3 is nowadays regarded to be a ubiquitous protein, the function of which is of crucial importance for all metazoan species; the family of 14-3-3 proteins comprises seven isoforms. An ELISA for quantifying 14-3-3 proteins in bodily fluids (serum, plasma, liquor cerebrospinalis, lacrimal secretion, urine) or in tissue/cell extracts from animals cannot be designed in a too sensitive form. Therefore, an ELISA has been developed, which is preceded by an enrichment method. It is known, that 14-3-3 proteins bind to the phosphorylated recognition sequence RSXpSXP. To this aim, the recognition sequence RSXpSXP is produced synthetically in a form having a biotin terminus (see FIG. 5). This sequence is added to wells of ELISA-plates, which were coated with streptavidin. After RSXpSXP has bound via biotin and streptavidin, 100 μl of bodily fluid (serum, plasma, liquor cerebrospinalis, lacrimal secretion, urine) or cell extract are added to the wells. After washing, the RSXpSXP-14-3-3 protein-complexes are quantified by means of labeled antibodies. As a label one can e.g. use peroxidase or alkaline phosphatase; suitable substrates for detection are then e.g. TMB or ABTS (2,2′-azinobis[3-ethylbenzothiazoline sulfonic acid]).

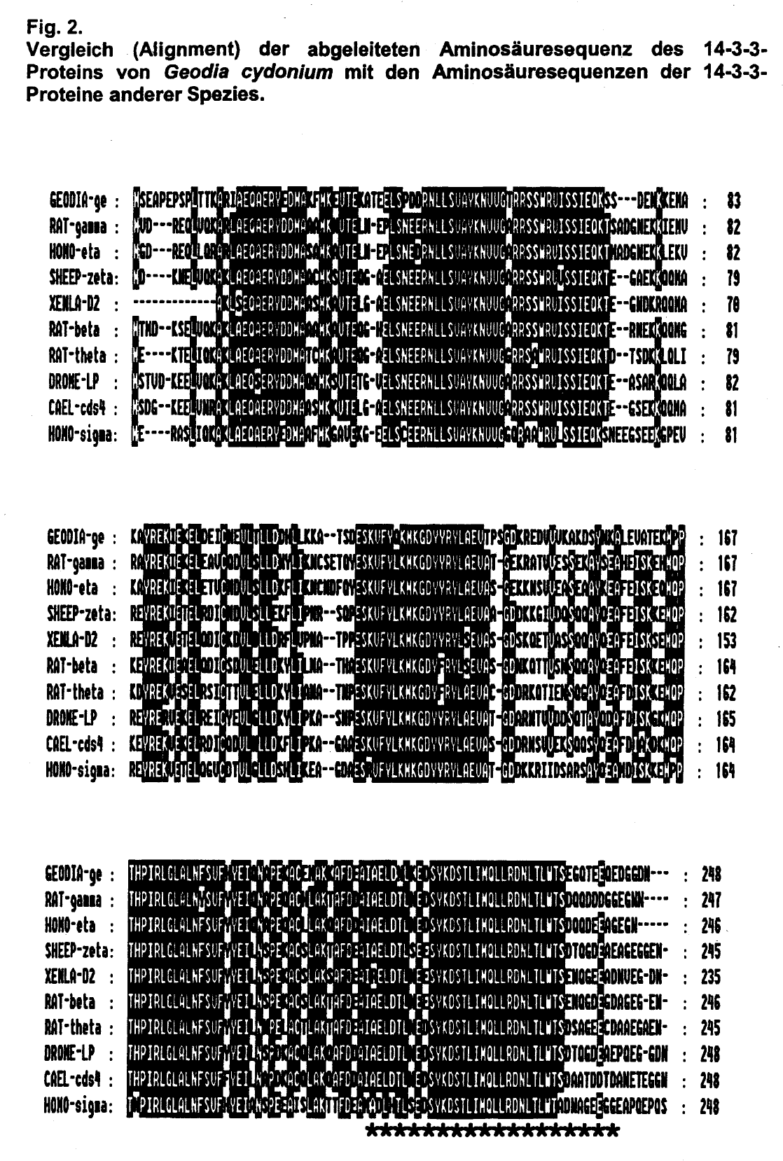

FIG. 2: A comparison (alignment) of the deduced amino acid sequence of the 14-3-3 protein of Geodium cydonium [GEODIA-ge] with the following isoforms of other species: gamma from the rat [RAT-gamma], eta from the human [HOMO-eta], zeta from the sheep [SHEEP-zeta], 14-3-3-related polypeptide from Xenopus laevis [XENLA-D2, 214097], beta from the rat [RAT-beta], theta from the rat [RAT-theta], Drosophila melanogaster 14-3-3 protein [DROME-LP], 14-3-3 protein from Caenorhabditis elegans [CAEL-cds4] and sigma from the human [HOMO-sigma]. Conserved amino acids in all sequences are presented in an inverted form; such amino acids, which are present in more than five sequences, are shown with a shaded background. The sequence employed for the generation of antibody is indicated (*****).

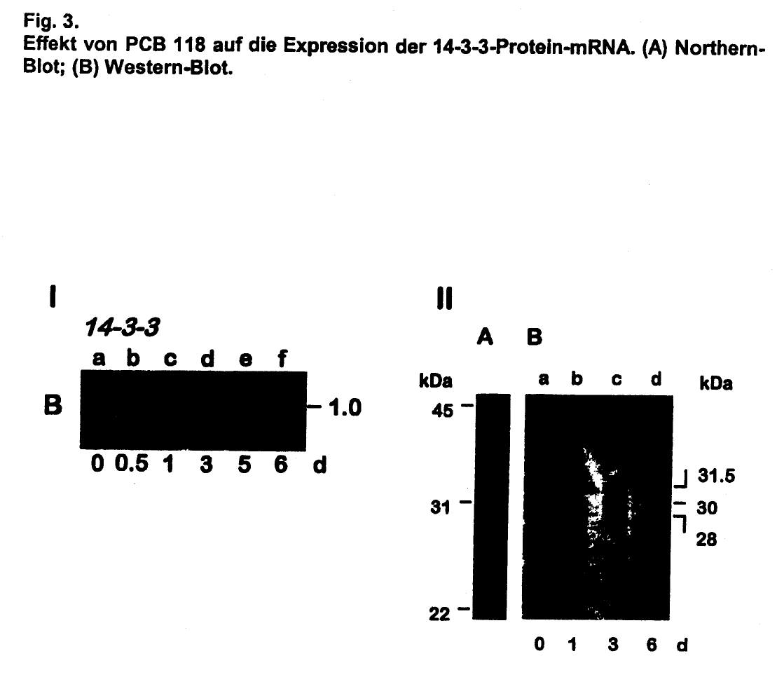

FIG. 3: The effect of PCB 118 on the expression of the 14-3 -3 protein mRNA after incubation of Geodium cydonium with 2 μg PCB 118/g wet weight for 0.5 to 6 days. The analyses were accomplished by means of Northern Blotting [14-3-3 probe] (A) or Western Blotting [14-3-3 antibody] (B).

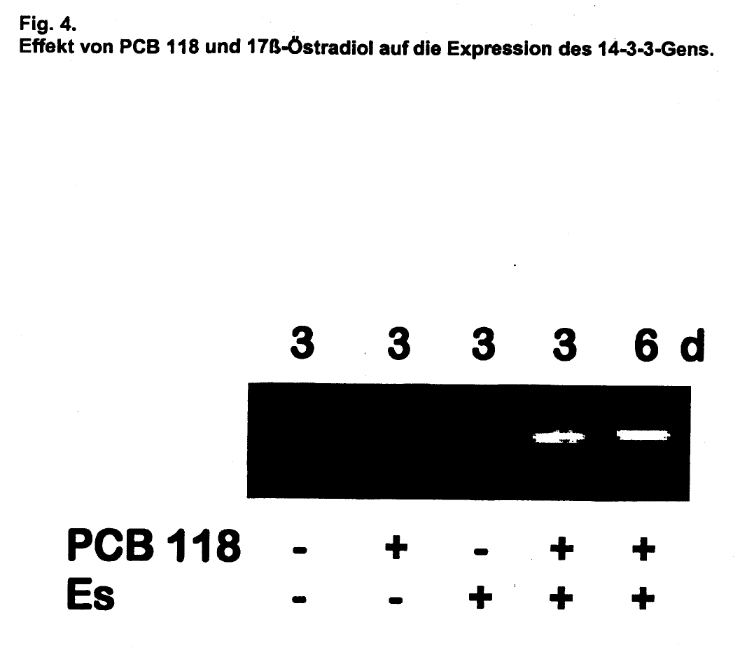

FIG. 4: The effect of PCB 118 (2 μg/g) and 17β-oestradiol (20 ng/g) on the expression of the 14-3-3 gen. The two chemicals were applied/added to the experimental animals (Geodium cydonium) either as individual substances or in combination. The incubation period was limited to 3 or 6 days.

FIG. 5: A schematic depiction of the principle of the developed ELISA-method for the quantitative detection of 14-3-3 (variant 2).

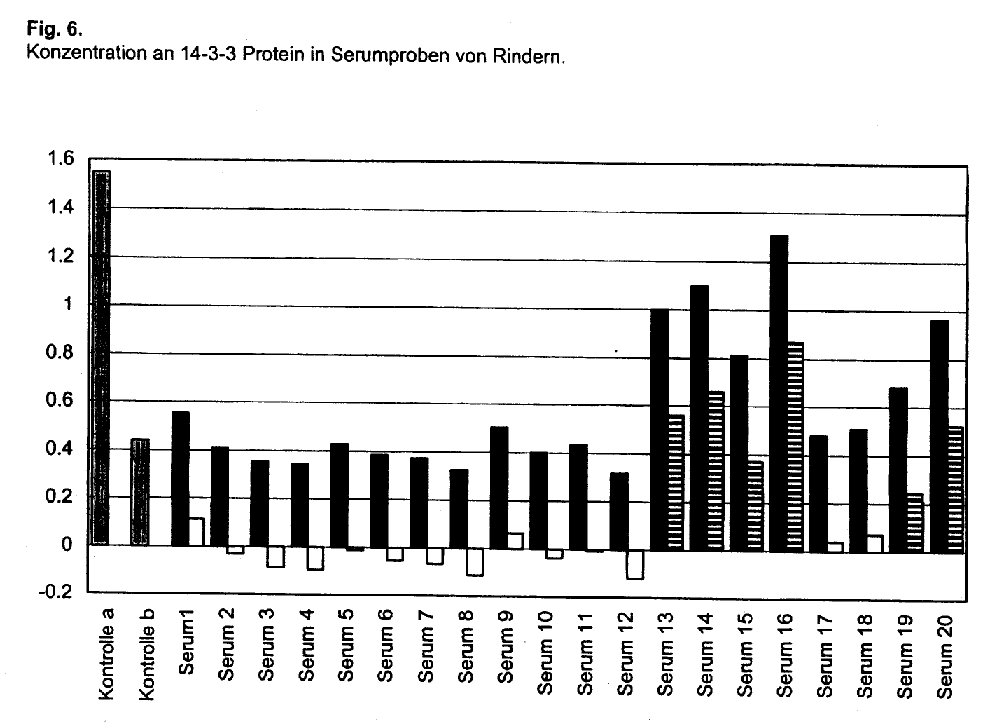

FIG. 6: The concentration of 14-3-3 protein in bovine serum samples (the y-axis shows the extinction, which is proportional to the concentration of 14-3-3 protein). Controls (grey bars): (a) without peptide and without blocking buffer and with both antibodies; (b) with peptide and with BSA blocking and with both antibodies. The black bars show the measured values, the white and the cross striped bars show the measured values after subtraction of control b. Double measurements were performed in each case. The sera 13, 14, 15, 16, 19 and 20 show an increased 14-3-3 protein level (cross striped bars).

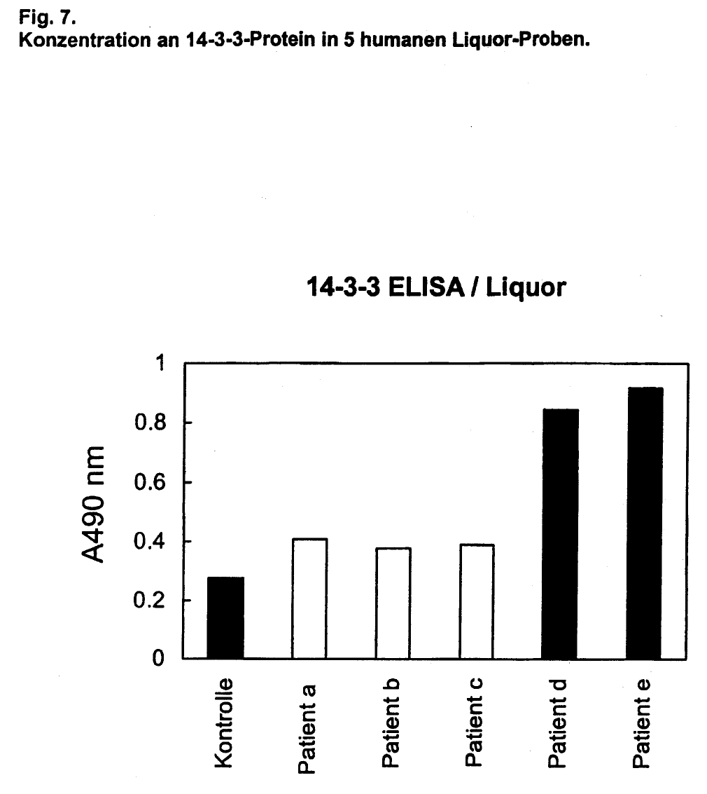

FIG. 7: The concentration of 14-3-3 protein in 5 human liquor cerebrospinalis samples (the y-axis shows the extinction, which is proportional to the concentration of 14-3-3 protein). The samples d and e (black bars) were derived from patients with Creutzfeldt-Jakob disease, the samples a-c (white bars) were derived from healthy persons. The grey bar represents the negative control.

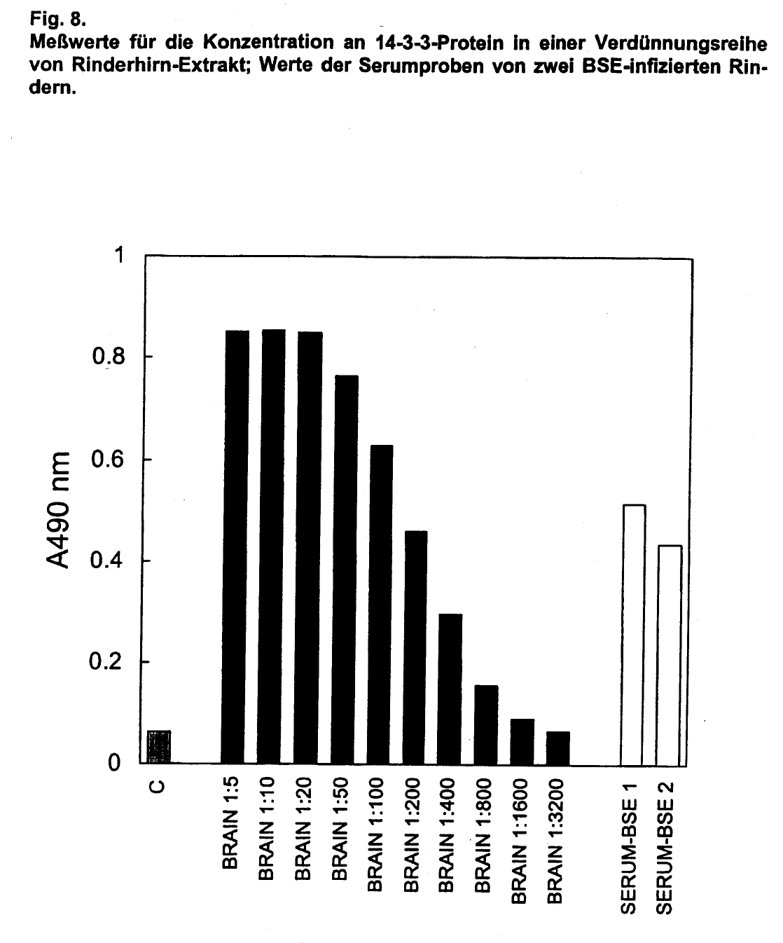

FIG. 8: The measured values for the concentration of 14-3-3 protein in a dilution series of bovine brain extract (black bars; the y-axis shows the extinction, which is proportional to the concentration of 14-3-3 protein). The indicated dilution steps correspond to the following amounts of 14-3-3 protein: 280 ng (dilution step 1:5); 140 ng (dilution step 1:10); 70 ng (dilution step 1:20); 28 ng (dilution step 1:50); 14 ng (dilution step: 1:100), 7 ng (dilution step: 1:200); 3.5 ng (dilution step 1:400); 1.75 ng (dilution step: 1:800); 0.88 ng (dilution step: 1:1600); and 0.44 ng (dilution step: 1:3200). As a comparison, the values for the serum samples of two BSE infected cows are shown (white bars). Negative control (grey bar).

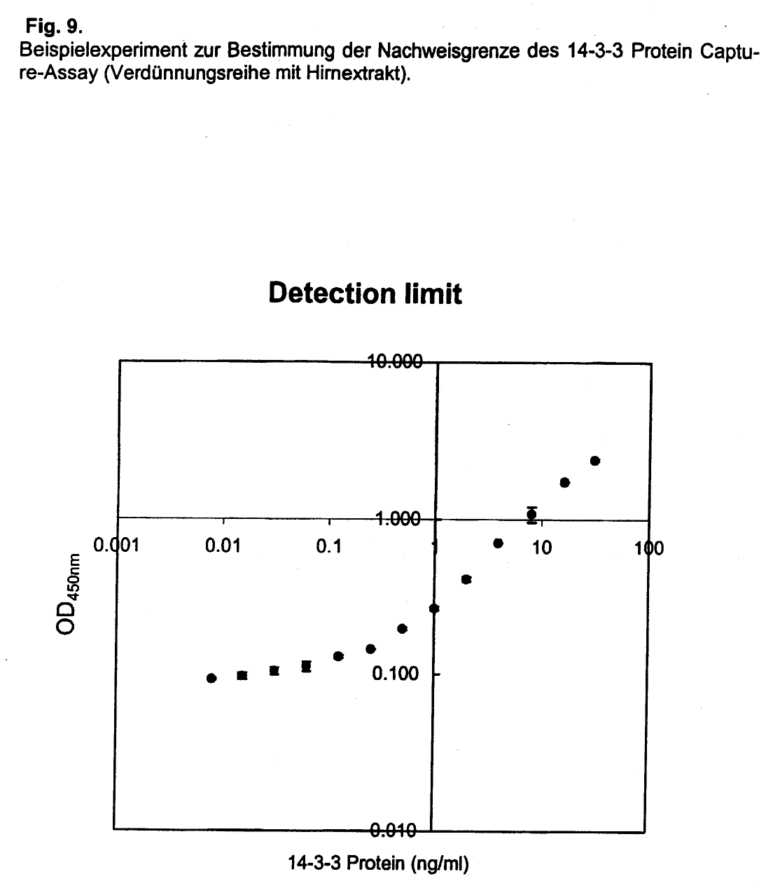

FIG. 9: The determination of the detection limit of 14-3-3 protein in the 14-3-3 protein capture assay. We tested a dilution series containing a brain extract with a known concentration of 14-3-3γ protein. The detection limit in the 14-3-3 protein capture assay was 0.02 ng/ml of 14-3-3γ protein (14-3-3 protein capture assay with TMB substrate).

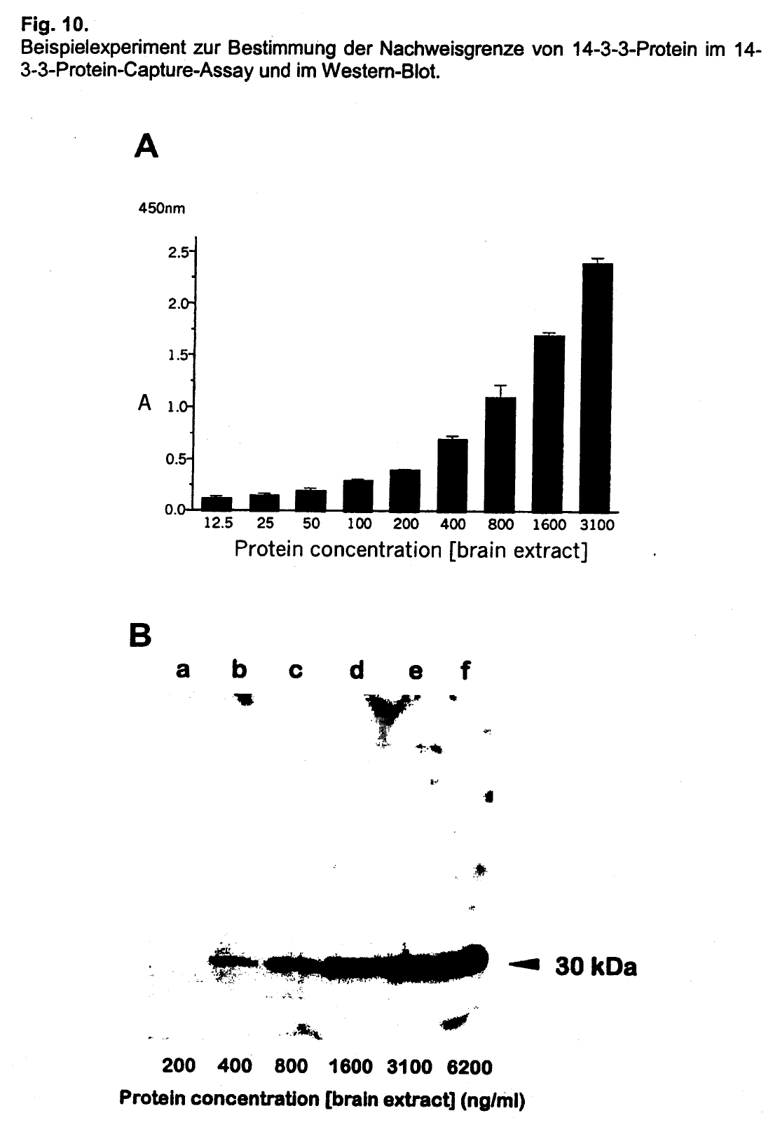

FIG. 10: The determination of the detection limit of 14-3-3 protein in the 14-3-3 protein capture assay and in the immunoblot. We tested a dilution series containing a brain extract with a known concentration of 14-3-3γ protein. The detection limit in the 14-3-3 protein capture assay was below 0.5 ng/ml of 14-3-3γ protein (0.02 ng/ml, as shown in FIG. 9). In comparison to this, the detection limit of the conventional Western Blot method was about 4 ng/ml of 14-3-3γ protein.

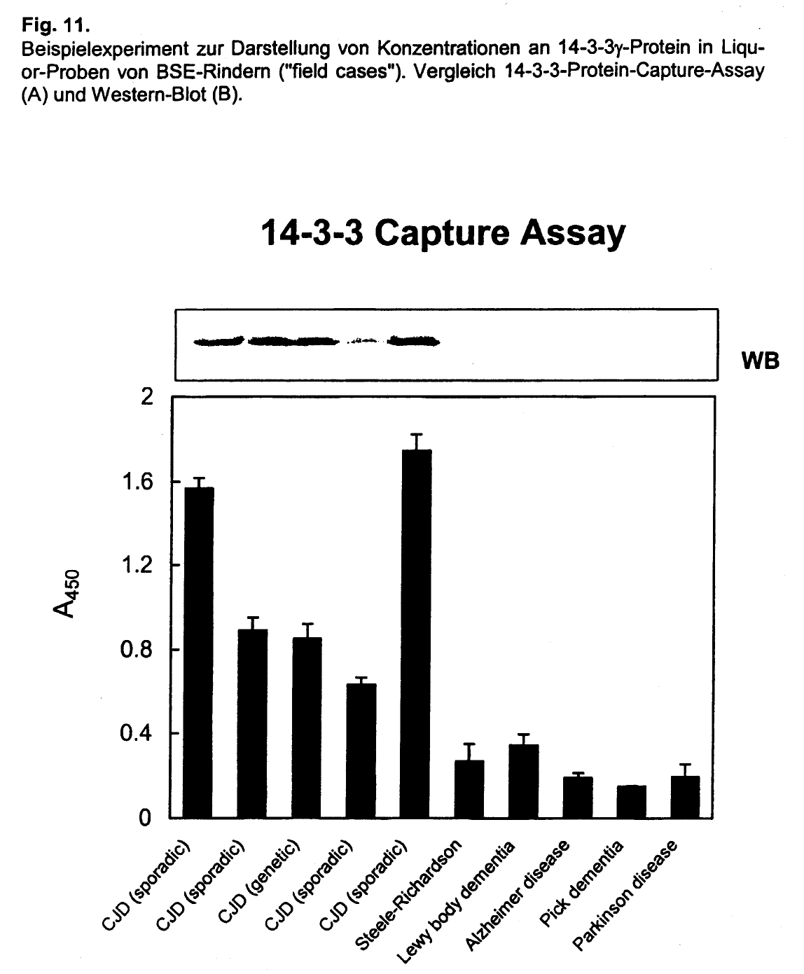

FIG. 11: The result of the representation of 14-3-3γ protein concentrations in liquor (cerebrospinal fluid; CSF) samples from 5 patients with CJD and from 5 patients suffering from other neuronal diseases (Multiple Sclerosis, Alzheimer's disease, Lewy Body dementia, Steele-Richardson's disease and Whipple's disease) by means of the 14-3-3 protein capture assay. In parallel, a Western Blot with a 14-3-3β antibody was performed. In contrast to the Western Blot, the 14-3-3 protein capture assay is able to also determine 14-3-3 protein in non-CJD-patients.

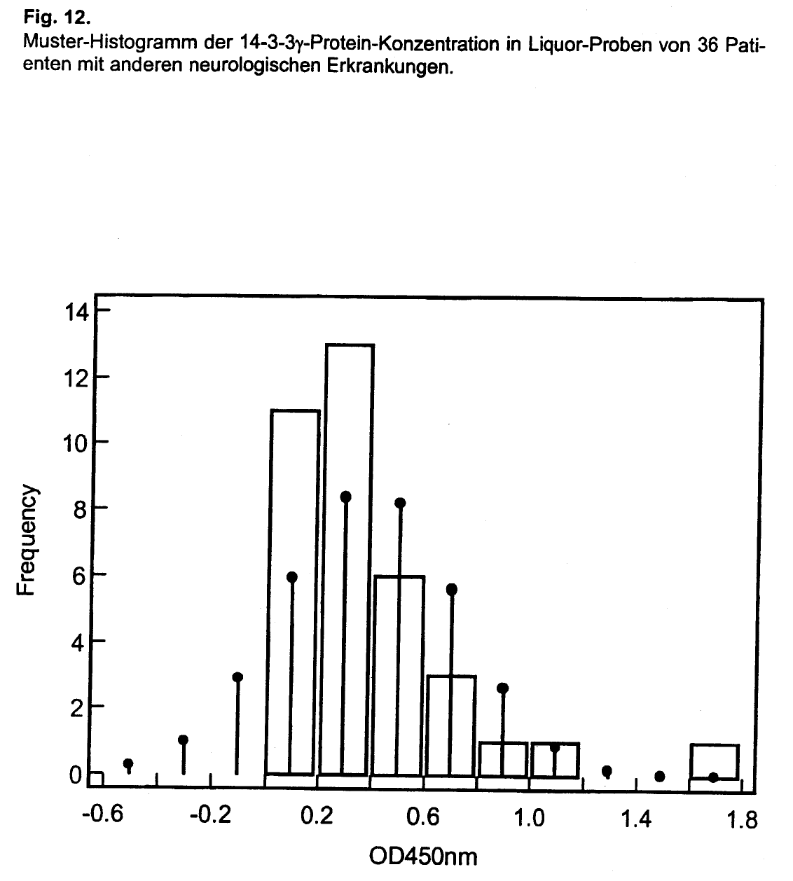

FIG. 12: A pattern histogram of the 14-3-3γ protein concentration in CSF samples from 36 patients with other neurological diseases including dementia (number: 9), Alzheimer's disease (7), encephalopathy (5), Lewy Body dementia (4), Lyme borreliosis (1), cerebellar syndrome (1), extrapyramidal syndrome (1), hydrocephalus (1), LEMP (1), lymphocytic meningitis (1), Parkinson's disease (1), SLA (1), Steele-Richardson's disease (1) and Wilson's disease (1). (14-3-3 protein capture assay with TMB substrate; Cut-off value: >0.98 OD_{450 nm} units). The normal distribution of the values represented in the histogram is shown in the form of vertical lines. By means of the 14-3-3 protein capture assay, it is—in contrast to the Western Blot—also possible to determine the distribution of the 14-3-3 protein concentrations in non-CJD patients.

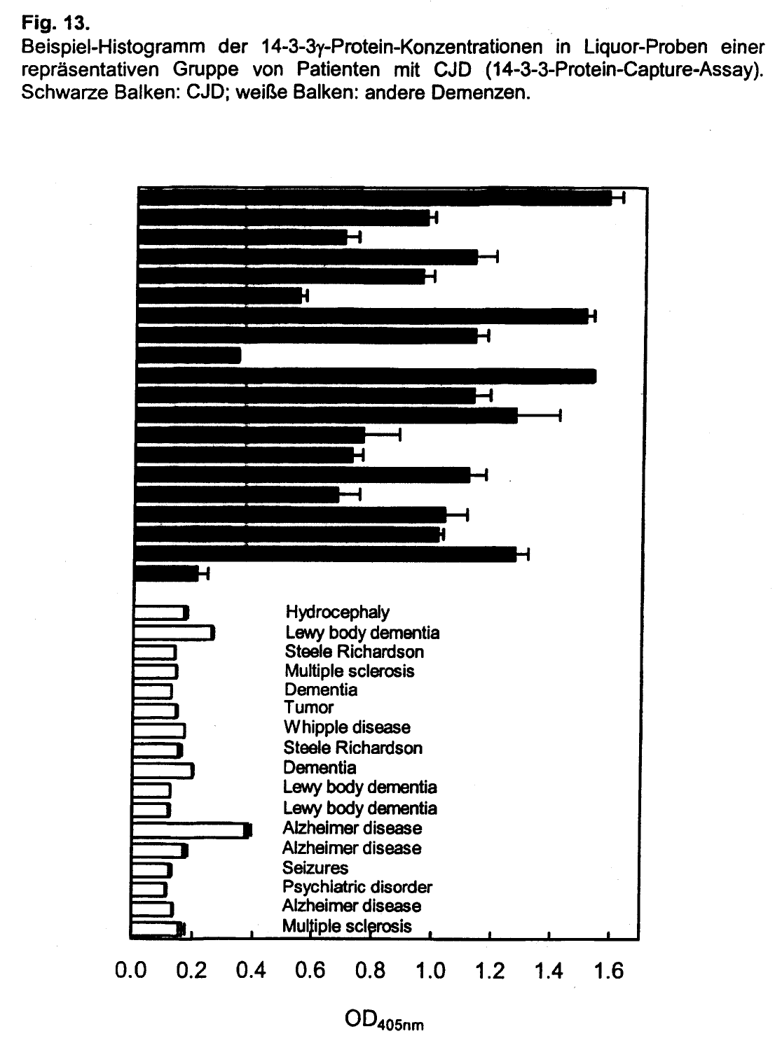

FIG. 13: The result of the determination of 14-3-3γ protein concentrations in liquor samples from a representative group of patients with CJD by means of the 14-3-3 protein capture assay. Tested was the liquor from 41 neuropathologically confirmed CJD patients (40 patients with sporadic CJD and one genetic CJD-patient having the PRNP V210I mutation and 36 patients with other neurological diseases like Alzheimer's disease, Huntington's disease, leucoencephalopathy, amyotrophic lateral sclerosis, Lewy Body dementia, Hashimoto's thyroiditis, vascular dementia, Pick's dementia, Parkinson's disease and alcoholic encephalopathy. The assay showed that the samples from CJD patients (liquor both from sporadic CJD and genetic CJD) contained significantly higher concentrations of 14-3-3γ protein than non-CJD samples (liquor from patients with other neurological diseases).

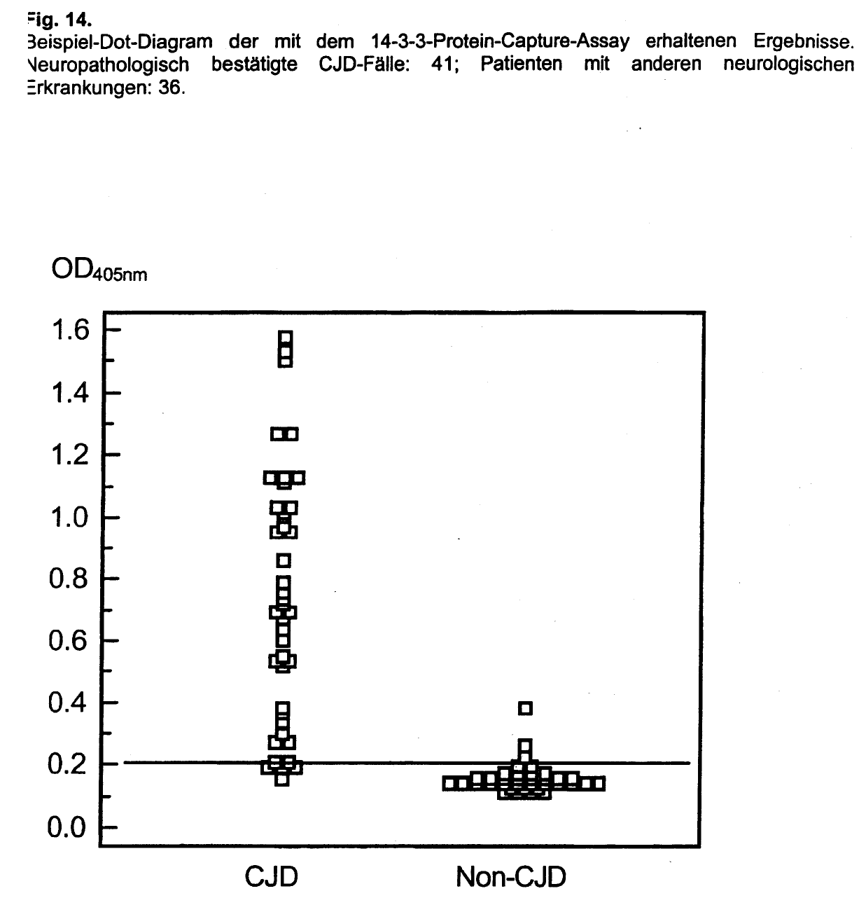

FIG. 14: A dot diagram of the results obtained for the 14-3-3 protein capture assay. What is shown are the OD_{450 nm} values both for the neuropathologically confirmed CJD cases (n=41) and for the non-CJD cases with other dementias (n=36). The Cut-off value (minimal number of false negative and false positive results) is indicated by a horizontal line.

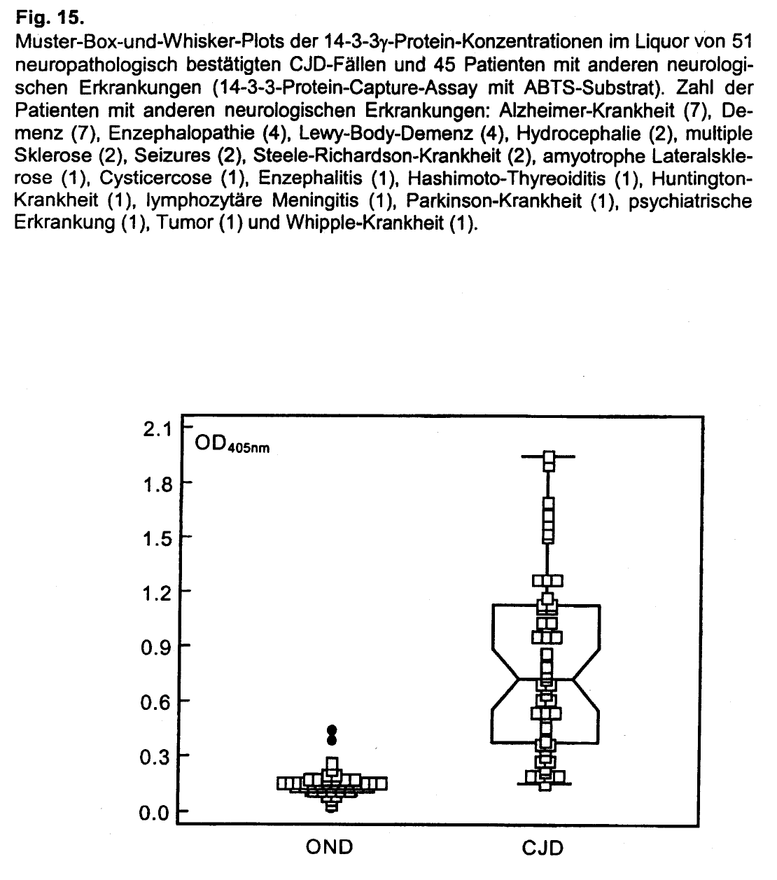

FIG. 15: The Box and Whisker plots of the 14-3-3γ protein concentrations in the liquor of 51 neuropathologically confirmed CJD cases and 45 patients with other neurological diseases (14-3-3 protein capture assay with ABTS substrate).

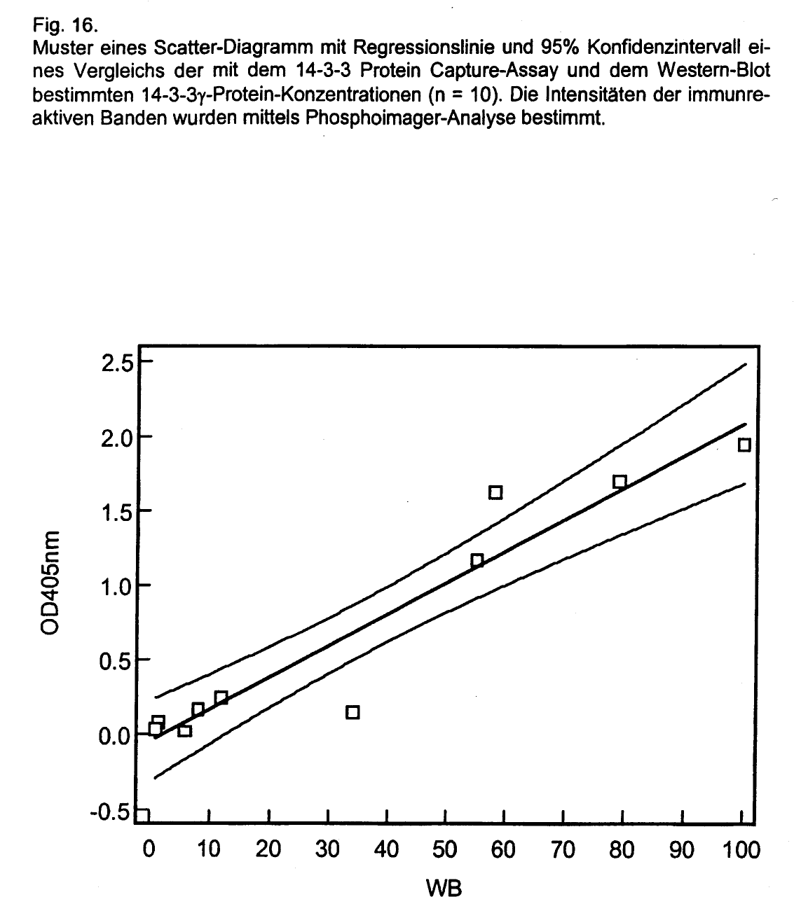

FIG. 16: A Scatter diagram of a comparison of the 14-3-3γ protein concentrations determined by means of the 14-3-3 protein capture assay and by the Western Blot. There is a very good correlation.

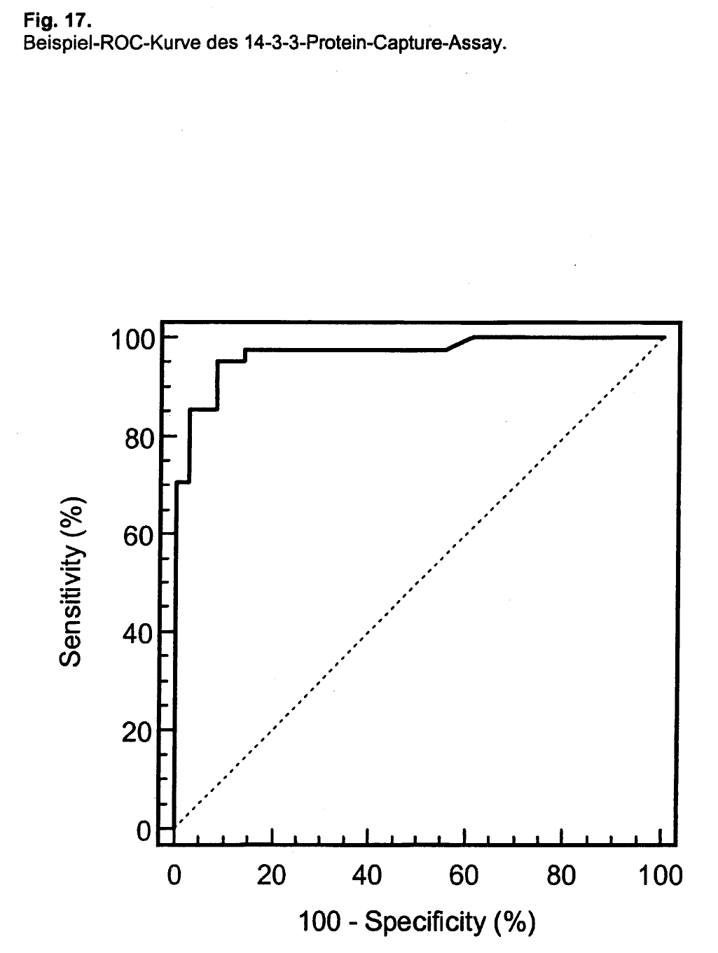

FIG. 17: The ROC-graph of the 14-3-3 protein capture assay. The sensitivity is plotted as a function of (100—specificity) for different Cut-off values.

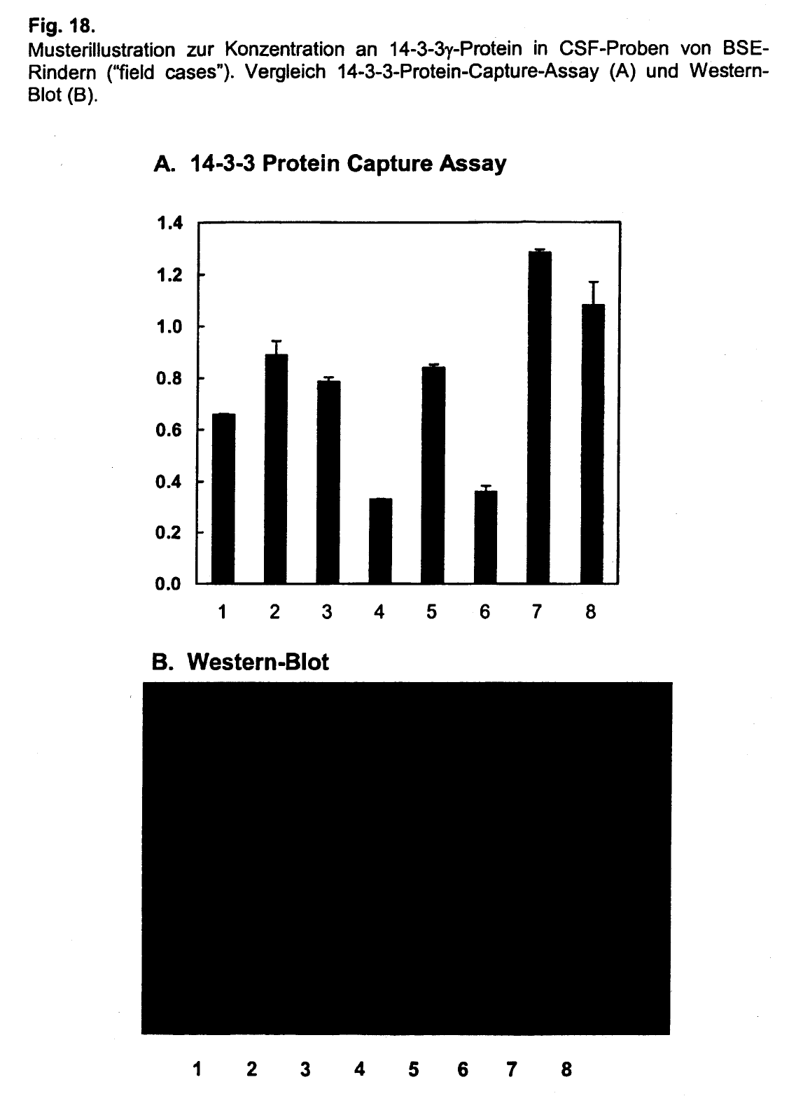

FIG. 18: The concentration of 14-3-3γ protein in liquor of BSE cows (“field cases”), comparatively determined by means of A.) 14-3-3 protein capture assay and B.) Western Blot. The comparison of results shows, that (1) the concentrations of 14-3-3γ protein in the capture assay are parallel to the band intensities in the Western Blot and (2) that the increased concentrations of 14-3-3γ proteins—in contrast to the 14-3-3 protein capture assay—are hardly detectable in the Western Blot.

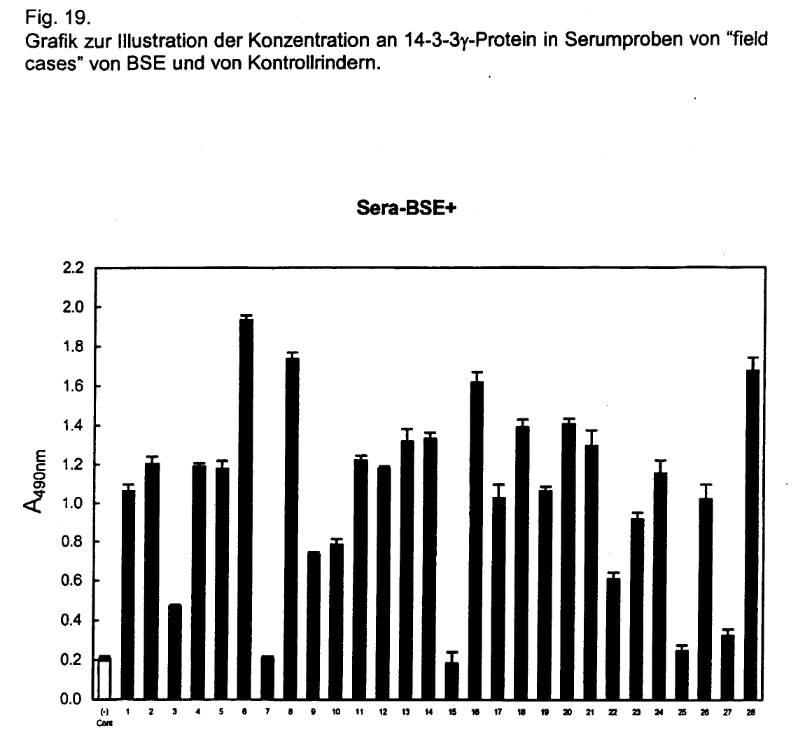

FIG. 19: The concentration of 14-3-3γ protein in the serum of BSE cows (“field cases”).

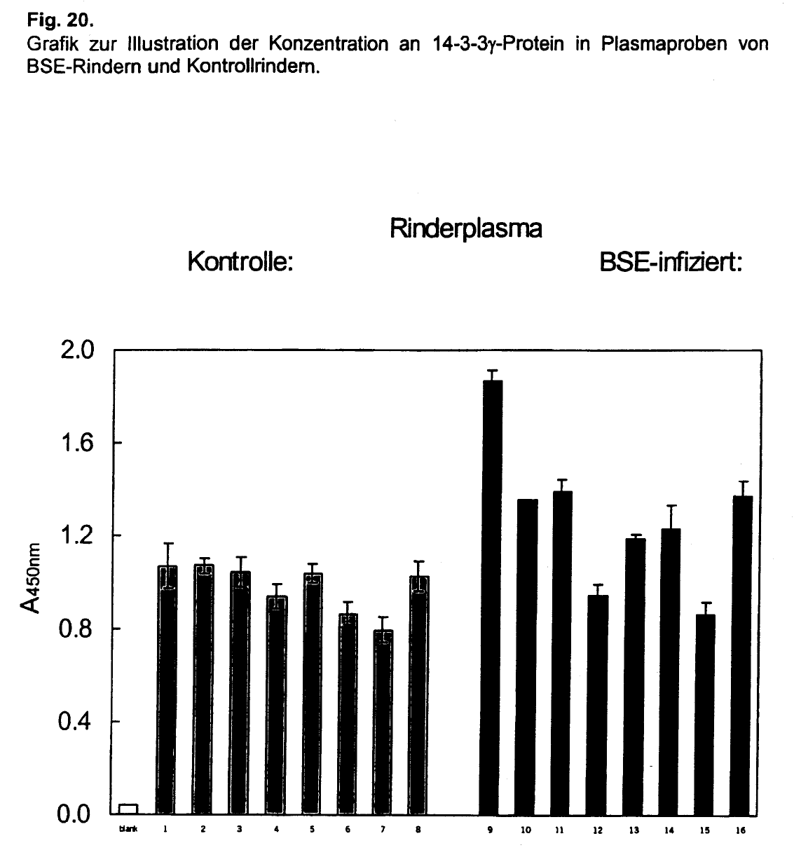

FIG. 20: The concentration of 14-3-3γ protein in the plasma samples from BSE cows and control cows.

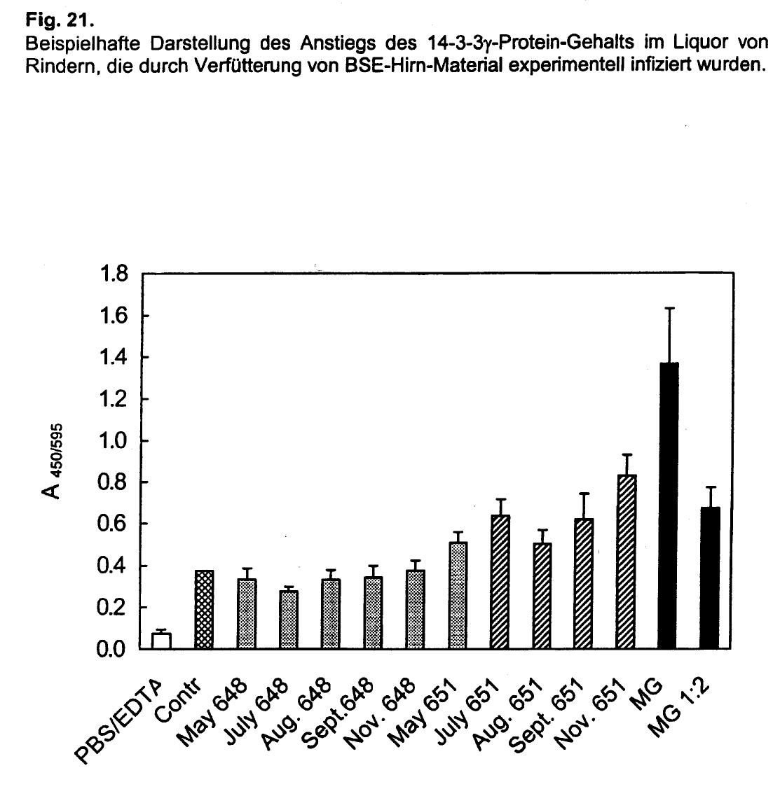

FIG. 21: An exemplary depiction of the increase of the 14-3-3γ protein level in liquor from BSE exposed cows, which were experimentally infected by feeding BSE brain material. Chequered bar: control cow; spotted bars: exposed cow no. 1; hatched bar: exposed cow no. 2; black bars: BSE cow (“field case”), undiluted liquor and 1:2 diluted liquor.

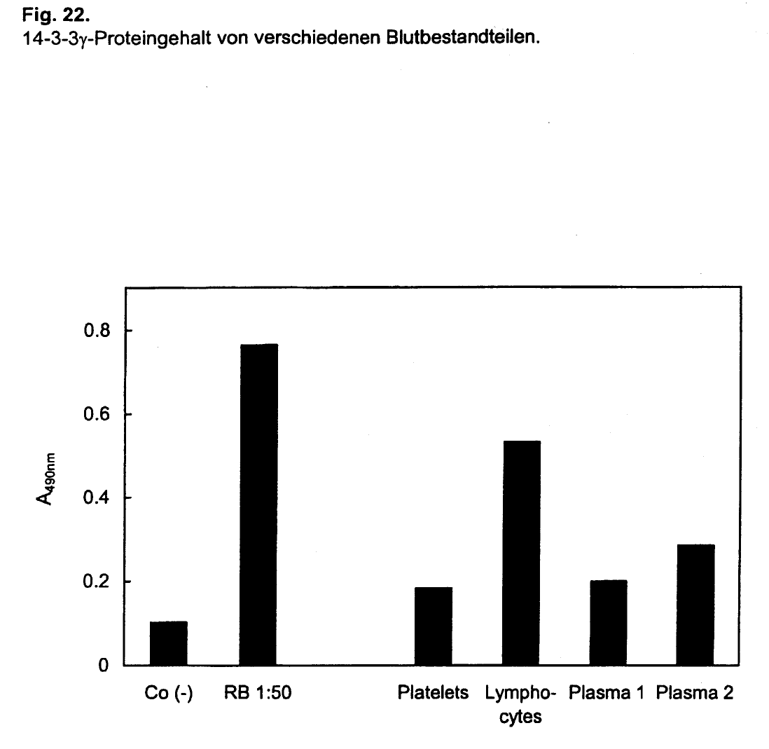

FIG. 22: The 14-3-3γ protein content of different blood components as an example. The protein concentration was 10 mg/ml. Plasma 1: lymphocyte-free plasma; plasma 2: cell-free plasma.

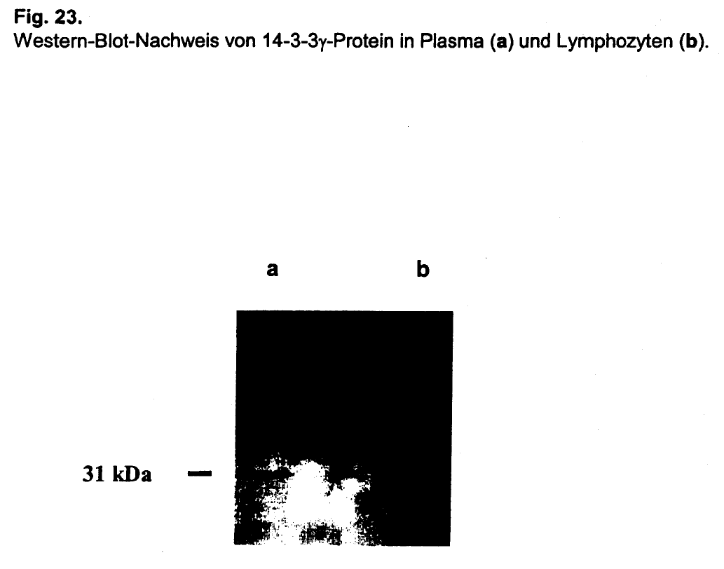

FIG. 23: A Western Blot detection of 14-3-3γ protein in plasma (a) and lymphocytes (b).

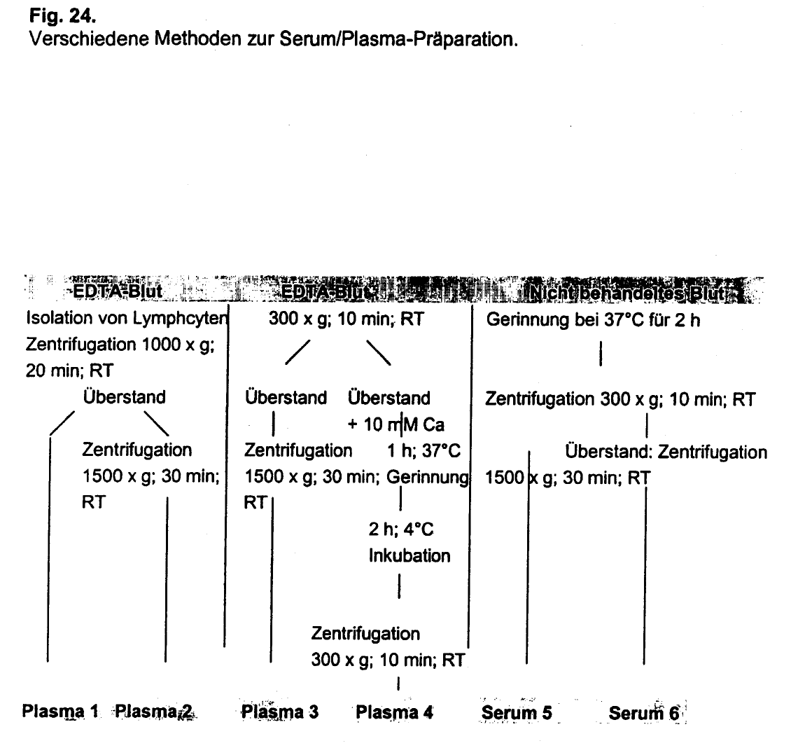

FIG. 24: Different methods for the preparation of serum/plasma; and

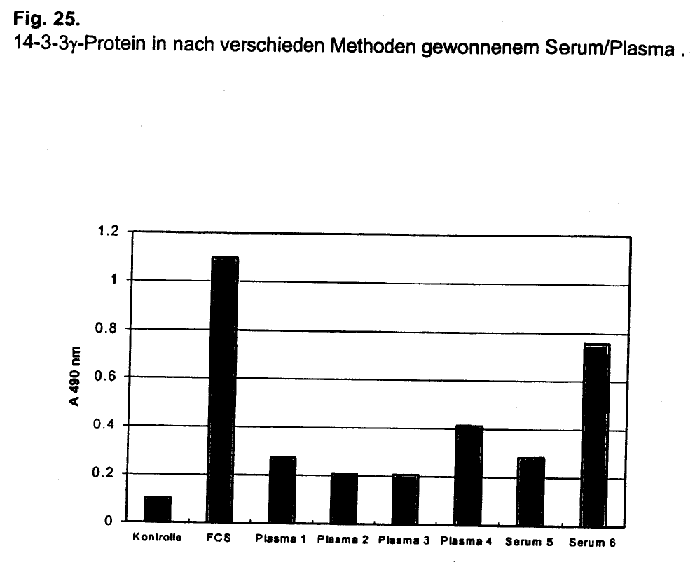

FIG. 25: The 14-3-3γ protein concentration in plasma/serum samples obtained by different methods as an example. Plasma 1: lymphocyte-free plasma (centrifugation by means of Uni-Sep tubes); Plasma 2: lymphocyte- and platelet-free plasma (separation of the lymphocytes by means of Uni-Sep tubes); Plasma 3: lymphocyte- and platelet-free plasma; Plasma 4: plasma obtained after coagulation; Serum 5: Serum without cellular components; Serum 6: cell-free serum.

The chemicals used in the experiments described in the following were obtained from the following companies: restriction endonucleases and other enzymes for recombinant DNA-techniques and vectors from Stragene (Heidelberg, Germany), QIAGEN (Hilden, Germany) and USB (Cleveland, Ohio, USA); corn oil (C8267) from Sigma (Deisenhofen, Germany); TRIzol Reagent from GibcoBRL (Grand Island, N.Y., USA); CDP [Di-sodium 2-chloro-5-(4-methoxyspiro{1,2-dioxetane-3,2′-(5 ′-chloro)-tricyclo[3.3.1.13,7]decane}-4-yl)phenylphosphate] from Boehringer Mannheim (Mannheim, Germany). The polyclonal antibody (rabbit-IgG; Cat.-No. PC70) directed against the conserved amino acid residues of the 14-3-3 protein family and the 14-3-3 peptides aa221 to aa242 were obtained from Calbiochem/Oncogene (Cambridge, Mass., USA). The anti-14-3-3γ antibody C-16 (Cat-No. sc-731) was obtained from the Santa Cruz Biotechnology company (Santa Cruz, Calif., USA). PCB 118 was obtained from the Dr. Ehrenstorfer GmbH (Augsburg, Germany). TMB-substrate solution was obtained from Roth (Karlsruhe, Germany).

Detection of the Expression of 14-3-3 Proteins on the mRNA-Level

Since large regions of the different 14-3-3 isoforms are strongly conserved (see FIG. 2), degenerated primers can be employed for identification and quantification of the mRNA. For the quantitative PCR analysis, the inventors already successfully employed a primer, directed against aa47-aa53; VAYKNVVG, 5′-GTKGCCTACAARAAYGTGGT-3′ [K=G/T, R=A/G and Y=C/T; forward primer].

For the quantification of the 14-3-3 protein mRNA by means of the Northern Blotting technique, gene probes, which extend from the conserved starting regions [aa 45-aa 57] up to the conserved end regions [aa 214-aa 234] of the 14-3-3 protein (FIG. 2) were constructed. The employed method was PCR cloning. As experience shows, the obtained 500 bp probes are well suited for Northern Blot analyses.

The quantitative evaluation of the signals can be accomplished e.g. by using the chemiluminescence method (Stanley and Kricka, Bioluminescence and chemiluminescence: current status. John Wiley & Sons, New York, 1990) in combination with a PhosphoImager (e.g. GS-525 Molecular Imager from the Bio-Rad company); [Di-sodium 2-chloro-5-(4-methoxyspiro{1,2-dioxetane-3,2 ′-(5′-chloro)-tricyclo[3.3.1.13,7]decane } -4-yl)phenylphosphate] (CDP) is used as a substrate for this.

Detection of the Expression of 14-3-3 Proteins on the Protein Level: Western Blotting

The detection of expression of the 14-3-3 proteins on the protein level is accomplished by using antibodies. To this aim, one can either employ commercial antibodies (e.g. from the Calbiochem/Oncogene company) or antibodies raised against the recombinant invertebrate-14-3-3 protein from Geodia cydonium. For the quantification of the 14-3-3 proteins, Western Blots with extracts of the bioindicator were produced. The signals can again be quantified by using the chemiluminescence method. The tissue extracts can e.g. be prepared by homogenisation in a phosphate buffer containing 1 mM of EDTA and 1 mM of phenylmethylsulfonyl fluoride.

### EXAMPLE 1

### Utilisation of 14-3-3 Proteins as a Biomarker for PCB: Detection by Means of Northern- and Western-Blotting

The 14-3-3-cDNA from the sea sponge Geodia cydonium was used as a gene probe for the detection of expression of the 14-3-3 proteins on the mRNA-level and the respective antibodies were used for determining the amount of protein. In the accomplished keeping experiments, PCB 118 was used as the model PCB.

### Realisation

(a) Exposition of G. cydonium towards PCB118:

1 ml of PCB118 (0.1 mg/ml in corn oil) were injected in 40 g sponge tissue. The samples were incubated in sea water for up to 6 days in 20 l aquariums at 17° C. with continuous aeration; the water was exchanged one time at day 2. Aliquot portions of the tissue (each about 200 mg) were withdrawn at the time zero or after an incubation period of 0.5; 1; 3; 5 and 6 days. These aliquot portions were immediately frozen in liquid nitrogen and stored at a temperature of −80° C.

(b) Extraction:

Extracts for the determination of the amount of 14-3-3 protein were obtained by crushing the frozen tissue samples with a mortar in a 3-fold volume of phosphate buffer, thereby adding 1 mM of EDTA and 1 mM of phenylmethylsulfonyl fluoride. RNA was extracted from the sponge tissue being pulverised in liquid nitrogen by the aid of the TRIzol Reagent (GibcoBRL) according to the manufacturer's instruction.

The keeping tests with G. cydonium showed, that PCB118 induces the expression of the 14-3-3 gene from “not detectable” to very high values (FIGS. 3A and B).

It has been impossible so far to detect comparably strong and unambiguous effects with any other biomarker for PCB in invertebrates. Moreover, it is striking and important for a potential use as a biomarker in other aquatic invertebrates that both the antibodies against the 14-3-3 protein of the sponge and the gene probe of G. cydonium cross-react/hybridise with the homologous proteins/RNAs of higher invertebrates and vertebrates.

### EXAMPLE 2

### Use of 14-3-3 Proteins As a Biomarker for (Xeno)estrogens: Detection by Northern Blotting

It could be shown by Northern Blotting, that both PCB and 17β-oestradiol strongly induce the expression of 14-3-3 in lower invertebrates (FIG. 4). Moreover, it was successfully shown, that an exponentiation of the expression of 14-3-3 protein mRNA sets in, when both substances are combined. In the absence of both agents, the 14-3-3 protein mRNA is not able to be detected. Whereas the level of the 14-3-3 protein mRNA was 30% when PCB118 is applied, and 100% (reference value) when 17β-oestradiol was applied, both chemicals together induced the expression of 14-3-3 protein at a value of 550%. The induction terminates after 3 days.

Detection of the Expression of 14-3-3 Proteins by Means of ELISA Methods (“14-3-3 Protein Capture Assay”)

Preparation of the ELISA Microtiter Plates

14-3-3 protein is a protein, which specifically binds to phosphoserine; a 14-3-3 protein binding motif has been identified (Muslin et al., Cell 84, 889-897, 1996). In the ELISA, which we have developed (“14-3-3 protein capture assay”), we coupled the peptide LPKINRSApSEPSLHR (pS=phosphoserine), to which the 14-3-3 protein binds with high affinity (Muslin et al., Cell 84, 889-897, 1996), N-terminally to a cysteine, whereat two alanine residues were used as a spacer.

Variant 1:

In this variant, the chemically synthesized peptide CAALPKINRSApSEPSLHR was covalently bound to Reacti-Bind™ maleimide activated microtiter plates (capacity: 100-150 pmol SH-containing peptides per well; Pierce company, Rockford, Ill., USA) via the reaction of the sulfhydryl group of the N-terminal cysteine. This was accomplished according to the manufacturer's instruction. Excess maleimide groups on the plates were blocked by incubating the plates with a cysteine solution (10 μg/ml) for 1 hour (alternatively: incubation with a 5% bovine serum albumin solution for 3 hours).

Before the coupling to the plates, the peptide solution was tested with Ellman's Reagent for the presence of SH-groups. If necessary (in case of an oxidation of the SH-groups with a formation of disulfide bonds), the peptide was treated with the ReduceImm Kit from the Pierce company (see above).

Variant 2:

In this alternative method, the chemically synthesized peptide CAALPKINRSApSEPSLHR was covalently bound to the EZ-Link™ biotinylation reagent Biotin-BMCC 1-biotinamido-4-[4′-(maleimidomethyl)-cyclohexanecarboxyamido]butane from the Pierce company (see above) via the reaction of the sulfhydryl group of the N-terminal cysteine. The binding of the biotinylated proteins/peptides to the microtiter plates in this case is accomplished via streptavidin (employment of streptavidin coated microtiter plates; FIG. 5).

Principal Procedure of the ELISAs

The procedure comprised the following steps:

Variant 1:

1a. Incubation of the streptavidin coated microtiter plates with the biotin-conjugated peptide.

2a. Addition of 100 μl extract from the investigated animal tissues to the wells after the peptide has been bound via biotin/streptavidin.

OR:

1a. Incubation of the extracts from the investigated animal tissues with the biotin-conjugated peptide.

2a. Addition of 100 μl of the incubation mixtures to the wells after the binding of the biotin-peptide to the 14-3-3 protein has been accomplished.

3. Washing.

4. Quantification of the peptide-14-3-3 protein-complexes by means of labeled antibodies. As a label, e.g. alkaline phosphatase may be used; a corresponding substrate for the detection in then 5-bromo-4-chloro-3-indolyl phosphate/nitroblue-tetrazolium.

5. Detection by means of an ELISA plate reader.

Variant 2:

1. Addition of 100 μl extract to the microtiter plate wells carrying the bound peptide; subsequent incubation.

2. Washing.

3. Quantification of the peptide-14-3-3 protein complexes by means of labeled antibodies (see variant 1).

4. Detection by means of an ELISA plate reader.

### EXAMPLE 3

### Quantification of 14-3-3 Proteins in Dabs after Exposition Towards PCBs and Cadmium. Detection by Means of the ELISA Method (14-3-3 Protein Capture Assay)

The amounts of PCB77, PCB 118 and PCB 153 given in table 1 were dissolved in corn oil and then injected into dabs (dabfish, Limanda limanda). The injection of the listed amounts of cadmium (see table 2) were injected as a solution in PBS. After the given periods, the liver was withdrawn from the animals and immediately frozen. The amount of 14-3-3 proteins in the livers was determined in a 3-fold volume of phosphate buffer with 1 mM EDTA and 1 mM of phenylmethylsulfonyl fluoride after the frozen tissue samples had been homogenised by the using a mortar.

TABLE 1
Injection [i.p.] of 0.03 mg PCB per kg of Limanda limanda
(dab, dabfish). After 5 days, the livers were withdrawn and
the amount of 14-3-3 protein determined by means of the
described ELISA. The optical densities are given in OD units.

| | 14-3-3 protein |
|---|---|
| Compound: | (OD unit) |
| PCB77 | 0.97 |
| PCB118 | 0.74 |
| PCB153 | 0.81 |
| Control | 0.12 |

TABLE 2
Injection [i.p.] of cadmium in Limanda limanda
(dab, dabfish). After 5 days the livers were withdrawn
and the amount of 14-3-3 proteins determined by means
of the described ELISA. The optical densities are given
in OD units.

| Cadmium | 14-3-3 protein |
|---|---|
| (mg/kg): | (OD unit) |
| 0.01 | 0.30 |
| 0.03 | 0.75 |
| 0.1 | 0.98 |
| Control | 0.12 |

14-3-3 Protein Capture Assay for the Diagnosis of Creutzfeldt-Jakob Diseases in the Human

The 14-3-3 protein capture assay constitutes a novel assay for the detection of the 14-3-3 proteins for early diagnosis both of the BSE disease in cattle and of the Creutzfeldt-Jakob disease in the human.

A test allowing for an early stage diagnosis of BSE or Creutzfeldt-Jakob disease from blood serum is not commercially available yet. The BSE-test developed by the Prionics company, which is based on specific antibodies, showed to be not applicable for serum samples and thus only allows for a post mortem diagnosis of these diseases. In contrast to this, the 14-3-3 protein capture assay allows for a diagnosis in living animals or patients. A protein binding assay for 14-3-3 protein (the principle of the assay described herein) has so far not been realised by any other group. A 14-3-3 protein ELISA, which is only based on antibodies, does not allow to be designed in a sufficiently sensitive form; the method described herein solves this problem by a specific enrichment of the protein (by means of an assay procedure, during which the 14-3-3 protein to be determined is bound to a specific binding peptide, which is coupled to a solid phase).

### Data Overview in the Use of 14-3-3 Proteins as a CJD Marker

Prion diseases:

(Transmissible spongiform encephalopathies, TSE)

fatal neurodegenerative diseases

infectious, hereditary or sporadic

bad prognosis

long incubation periods

They are characterised by:

spongiform degeneration of the brain

reactive gliosis

loss of nerve cells

accumulation of an abnormal isoform (PrP^{Sc}) of the cellular prion protein (PrP^{C}) in the brain

Human prion diseases:

Creutzfeldt-Jakob disease (CJD)

Gerstmann-Sträussler-Scheinker syndrome (GSS)

Fatal familial insomnia (FFI)

Kuru

GSS and FFI: Mutations in the PrP gene, which is localised on the short arm of chromosome 20.

Four variants of CJD:

sporadic

genetic

iatrogenic

novel variant (vCJD)

sporadic CJD: 85% of all cases of human prion diseases

incidence of CJD: ˜1 case per 1 million per year

Diagnosis of CJD:

a final diagnosis of sporadic CJD and vCJD can currently only be made post mortem by verifying the presence of PrP^{Sc} in the brain tissue by means of neurohistopathological methods

clinical criteria like a rapidly proceeding dementia, ataxia, myoclonus, and changes in the EEG only allow for a probable, but not for a definite diagnosis of CJD

Liquor proteins, which are potentially useful as a marker for the nerve cell damage in CJD patients:

14-3-3 Proteins

S-100 Protein

neuron-specific Enolase

Tau protein

Among these markers, the 14-3-3 proteins showed the highest specificity.

### EXAMPLE 4

### Quantification of One or More 14-3-3 Protein Isoforms in Bovine Serum and Liquor Cerebrospinalis. Detection by Means of an ELISA Method (14-3-3 Protein Capture Assay).

In prion diseases (BSE in cattle, Scrapie in sheep, Creutzfeldt-Jakob disease in the human, etc.) there is an increase in the concentration of 14-3-3 protein in the liquor cerebrospinalis (cerebrospinal fluid) (Jones et al., Veterinary Record 139, 360-363, 1996; Lee and Harrington, Electrophoresis 18, 502-506, 1997; Lee and Harrington, Veterinary Record 140, 206-207, 1997; Zerr et al., Ann. Neurol. 43, 32-40, 1998).

In the following assay, the ELISA method (14-3-3 protein capture assay) was used to determine the concentration of 14-3-3 proteins in different bovine serum samples (cattle fallen ill with BSE and non-infected controls) and in liquor cerebrospinalis (patients with Creutzfeldt-Jakob disease).

Performance:

1. Over night incubation of a maleimide activated microtiter plate (Reacti-Bind™ from the Pierce company; Cat-No. 15150) with 100 μl peptide solution {100 μg/ml CAALPKINRSApSEPSLHR and 60 μM TCEP.HCl [Tris(2-carboxyethyle)phosphine.HCl; Pierce company] in PBS/1 mM EDTA (pH 7.0; PBS=phosphate buffered saline solution consisting of 1.15 mM KH₂PO₄, 8.1 mM Na₂HPO₄, 137 mM NaCl and 2.7 mM KCl)} per well at room temperature and with gentle shaking.

2. Washing 4 times with 200 μl PBS (pH 7.4) per well

3. Blocking free maleimide groups on the plate in an 3 hour incubation period at room temperature with 200 μl of 3% bovine serum albumin (BSA), 10 μg/ml cysteine and 120 μM of TCEP.HCl in PBS/1 mM EDTA (pH 7.0).

4. Shaking out the plate and washing 4 times with 200 μl PBS (pH 7.4) per well.

5. Incubation with 100 μl sample solution (liquor cerebrospinalis or liquor dilution, or serum or serum dilution, or plasma or plasma dilution) per well for 1 hour at room temperature.

6. Washing 4 times with PBS (as indicated above).

7. Addition of an anti-14-3-3 protein antibody (e.g. an anti-14-3-3γ antibody; Cat-No. sc-731 from the Santa Cruz Biotechnology company; dilution 1:2000) in PBS (pH 7.4, containing 3% BSA) and incubation for 2 hours at room temperature on the shaker; subsequently washing 4 times with PBS (as above).

8. Addition of 150 μl peroxidase-conjugated anti-rabbit immunoglobulin (dilution 1:2000) in PBS (pH 7.4, containing 3% BSA) per well and incubation for 1 hour at room temperature; subsequently washing 4 times with PBS (as above).

9. Addition of 100 μl substrate buffer per well [substrate buffer: 1 ml of a 0.4% ortho-phenylenediamine (OPD) solution plus 9 ml substrate buffer plus 10 μl H₂O₂].

10. Determination of the optical density at 490 nm in an ELISA-reader after about 10 minutes.

Examples of controls:

a) without peptide and without blocking buffer with both antibodies

b) with peptide and with BSA-blocking with both antibodies

FIG. 6 shows, that the concentrations of 14-3-3 protein determined by means of the ELISA method (14-3-3 protein capture assay) are increased in the bovine serum samples 13, 14, 15, 16, 19 and 20, but not in the controls.

The ELISA method allows to detect also the increased concentration of 14-3-3 proteins in the liquor cerebrospinalis (cerebrospinal fluid) samples of patients with Creutzfeldt-Jakob disease. As it is shown in FIG. 7, the 14-3-3 protein levels in the liquor cerebrospinalis from two patients with Creutzfeldt-Jakob disease (patients d and e) are significantly higher than the respective levels of healthy control persons (patients a-c).

### EXAMPLE 5

### Quantification of One or More 14-3-3 Protein Isoforms in the Bovine Brain. Analysis of the Sensitivity of the ELISA Method (14-3-3 Protein Capture Assay).

In order to determine the sensitivity of the ELISA method, a dilution series of a bovine brain extract having a known concentration of 14-3-3 protein was produced and the respective 14-3-3 content measured by means of the ELISA method (14-3-3 protein capture assay). As it is shown in FIG. 8, the detection limit of 14-3-3 protein is to be found at about 1 ng (sample volume: 100 μl), thus at a concentration of about 10 ng/ml of 14-3-3 protein. The detection limit can still be further improved, e.g. to less than 1 ng/ml by a later stop of the dye reaction. The 14-3-3 protein concentrations being present in the sera (FIG. 8) or liquor (not depicted in FIG. 8) of BSE infected cattle are within the measuring range of the ELISA method.

The detection limit of the conventional Western Blot method however is about 40 ng/ml; this method is thus much less sensitive than the ELISA method, which we have developed.

A problem of the method described in the patent application WO 99/46401 was the yet insufficient detection limit (1-10 ng/ml). The specificity and sensitivity of the assay was able to be realised according to the invention by decreasing the concentration of the peptide solution and by the replacement of TCEP and cysteine in the manufacture of the plates and also by the addition of Tween20 to the solutions. These modifications of the protocol now make it possible to perform a trustworthy determination of 14-3-3 protein from human or animal also in the lower range of concentration. The modified method is as follows:

Preparation of the Plates for the 14-3-3 Protein Capture Assay

1. Over night incubation of maleimide activated microtiter plate (Reacti-Bind™ from the Pierce company; Cat-No. 15150) with 100 μl peptide solution {10 μg/ml CAALPKINRSApSEPSLHR in PBS/1 mM EDTA (pH 7.0; PBS=phosphate buffered saline solution consisting of 1.15 mM KH₂PO₄, 8.1 mM Na₂HPO₄, 137 mM NaCl and 2.7 mM KCl)} per well at room temperature and with gentle shaking.

2. Siphoning off the peptide solution or beating it out on an absorbent underlay; washing 4 times with 200 μl of PBS (pH 7.0; 0.05% Tween20) per well.

3. Blocking free maleimide groups on the plate by a 3 hour incubation period at room temperature with 200 μl of 3% bovine serum albumin (BSA) in PBS/1 mM EDTA (pH 7.0).

4. Siphoning off the blocking buffer or beating it out on an absorbent underlay; allowing the plate to dry at room temperature for 2 hours.

5. Shrink-wrapping the plate under vacuum; storage at −20° C.

Performance of the 14-3-3 Protein Capture Assay

1. Incubation with 100 μl sample solution (liquor cerebrospinalis or liquor dilution, or serum or serum dilution, or plasma or plasma dilution) per well for 1 hour at room temperature.

2. Washing 4 times with 200 μl washing buffer (PBS/0.05% Tween20; pH 7.0) per washing.

3. Addition of an anti-14-3-3 protein antibody (e.g. an anti-14-3-3γ antibody; Cat-No. sc-731 from the Santa Cruz Biotechnology company; dilution 1:2000) in PBS (pH 7.4, containing 3% BSA) and incubation for 2 hours at room temperature; subsequently washing 4 times with washing buffer (as above).

4. Addition of 150 μl peroxidase-conjugated anti-rabbit immunoglobulin (dilution 1:2000) in PBS (pH 7.0; containing 3% BSA, 1 mM EDTA) per well and incubation for 1 hour at room temperature; subsequently washing 4 times with PBS (as above).

5. Addition of 100 μl TMB substrate solution per well.

6. Allowing an incubation period of about 10 minutes; then adding 50 μl of 1M H₂SO₄ stopping solution per well by pipetting and mixing by gentle shaking.

7. Determining the optical density at 450 nm in an ELISA-reader (if possible with a reference wavelength of 595 nm).

The sensitivity in respect to the solution of the 14-3-3 protein capture assay can be increased by the employment of a peroxidase-labeled secondary antibody and a TMB-substrate in comparison to the employment of an alkaline phosphatase-labeled secondary antibody and the ABTS-substrate by a factor >4.

Moreover, a quality control of the analysis is made possible by the incorporation of a second marker/indicator assay. The prevention of a contamination of the liquor samples with blood can be achieved by combining the 14-3-3 protein capture assay with the haemoglobin peroxidase reaction with o-tolidine, tetramethylbenzidine or the like.

A quantification of the 14-3-3 protein capture assay is possible when combining the assay with a standard (recombinant 14-3-3 protein or standardised dilution of brain extract having a known 14-3-3 protein concentration).

A specificity test of the 14-3-3 protein capture assay is possible when using a competition with recombinant 14-3-3 protein (addition of increasing amounts of recombinant 14-3-3 protein to the assay).

The detection limit—when using the above described modified procedure for the 14-3-3 protein capture assay—is about 0.02 ng/ml for the 14-3-3γ protein; as a comparison: the detection limit for the Western Blot, performed with a highly sensitive chemiluminescence method: 4 ng/ml for the 14-3-3γ protein [FIG. 9: exemplary experiment for determining the detection limit of the 14-3-3 protein- capture assay (TMB substrate). FIG. 10: Exemplary example for determining the detection limit for 14-3-3 protein in the 14-3-3 protein capture assay and in the Western Blot].

The 14-3-3 protein capture assay was successfully tested with Creutzfeldt-Jakob patients. Liquor samples of Creutzfeldt-Jakob patients in comparison to liquor samples from patients with other brain diseases showed significantly higher values for the 14-3-3 protein (FIG. 11: exemplary experiment for representing the concentrations of 14-3-3γ protein in liquor samples from 5 patients with CJD and 5 patients with other neuronal diseases). In contrast to the Western Blot, the 14-3-3 protein capture assay is also capable to detect the 14-3-3 protein in non-Creutzfeldt-Jakob-patients.

However, the currently used immunoblotting methods do not allow to measure the generally low concentrations of this protein in the liquor of non-Creutzfeldt-Jakob-patients. The 14-3-3 protein capture assay for reason of its high sensitivity is also capable to determine the distribution of the 14-3-3 protein concentration in non-Creutzfeldt-Jakob-patients (FIG. 12: pattern histogram of the 14-3-3γ protein concentration in liquor samples from 36 patients with other neurological diseases).

Furthermore, the currently used immunoblotting (Western Blot-) methods do not allow to determine slight alterations of the 14-3-3 protein concentration in the liquor from patients with iatrogenic Creutzfeldt-Jakob disease or vCJD (novel variant of the Creutzfeldt-Jakob disease). Due to its high sensitivity, the 14-3-3 capture assay allows to accomplish this measurement.

It is known, that a trustworthy diagnosis of the Creutzfeldt-Jakob disease is often difficult when merely being based on clinical symptoms and alterations of the EEG. Our results have proven that the 14-3-3 protein capture assay is a suitable test for the pre-mortem diagnosis of the Creutzfeldt-Jakob disease.

The 14-3-3 protein capture assay offers a series of advantages in comparison to conventional immunoblotting methods:

The assay is quick (5 hours) and easy to perform.

It is simple and less susceptible to misinterpretations than the Western Blot.

It is highly sensitive and allows to detect slight alterations of the 14-3-3 protein concentration.

It allows for a quantification of the 14-3-3 protein.

It does not require to concentrate the samples

By means of the 14-3-3 protein capture assay it was able to be shown that liquor samples from Creutzfeldt-Jakob patients, namely also including sporadic and genetic Creutzfeldt-Jakob diseases, contain higher concentrations of 14-3-3γ protein in comparison to the samples from non-Creutzfeldt-Jakob patients (FIG. 13: exemplary histogram of the 14-3-3γ protein concentrations in liquor samples from a representative group of CJD-patients and non-CJD-patients, determined by means of the capture assay). The concentration of the γ-isoform is specifically increased in Creutzfeldt-Jakob patients.

A dot diagram of the results is shown in FIG. 14 (exemplary dot diagram of the results obtained by means of the 14-3-3 capture assay. neuropathologically confirmed CJD-cases: 41; patients with other neurological diseases: 36).

Table 3 shows a comparison of the results obtained by means of the 14-3-3 protein capture assay and the Western Blot.

TABLE 3
Comparison of the results obtained by the capture assay
and by the Western Blot technique

| | Capture Assay | Western Blot | |
|---|---|---|---|
| | (n/n oder %)¹ | (n/n oder %)² | (n/n oder %)³ |
| Positive | 39/41 (95%) | 38/41 (93%) | 40/41 (98%) |
| False negative | 2/41 (5%) | 3/41 (7%) | 1/41 (2%) |
| Negative | 33/36 (92%) | 36/36 (100%) | 36/36 (100%) |
| False positive | 3/36 (8%) | 0/36 (0%) | 0/36 (0%) |

¹With the selected Cut-off value of >0.20 OD_{405 nm}.
²Questionable Western Blot results (+/−) were evaluated as negative (−).
³Questionable Western Blot results (+/−) were evaluated as positive (+).

With the selected Cut-off value of >0.20 OD_{405 nm}, false positive results (with an OD_{405 nm} of 0.20-0.373; see exemplary dot diagram of FIG. 14) were observed in three non-Creutzfeldt-Jakob cases (two cases of Alzheimer's disease and one case of Lewy Body dementia). Only two neuropathologically confirmed Creutzfeldt-Jakob cases showed false negative results (OD_{405 nm} of 0.154 and 0.195). Both cases also gave questionable results (+/−) in the Western Blot.

FIG. 15 shows pattern Box-and-Whisker plots of the 14-3-3γ protein concentrations in the liquor from 51 neuropathologically confirmed CJD-cases and 45 patients with another neurological disease (14-3-3 protein capture assay with ABTS substrate). Numbers of the patients with other neurological diseases: Alzheimer's disease (7), dementia (7), encephalopathy (4), Lewy Body dementia (4), hydrocephalus (2), Multiple Sclerosis (2), seizures (2), Steele-Richardson's disease (2), amyotrophic lateral sclerosis (1), cysticercosis (1), encephalitis (1), Hashimoto's thyroiditis (1), Huntington's disease (1), lymphocytic meningitis (1), Parkinson's disease (1), psychiatric disease (1), tumour (1) and Whipple's disease (1).

FIG. 16 shows the correlation of the results of the 14-3-3 protein capture assay with the Western Blot for Creutzfeldt-Jakob patients under employment of 14-3-3γ isoform specific antibodies. Patients with sporadic Creutzfeldt-Jakob disease as well as patients with genetic Creutzfeldt-Jakob disease were investigated. A comparison of the results obtained by means of the capture assay with those of the Western Blot showed a high degree of conformity (FIG. 16: pattern of a Scatter diagram with regression line and 95% confidence interval of a comparison of the 14-3-3γ protein concentrations (n=10) determined by means of the 14-3-3 protein capture assay and by the Western Blot. The intensities of the immunoreactive bands were determined by Phosphoimager analysis.) A comparison of the results for the patients with non-Creutzfeldt-Jakob dementias is not possible, because the Western Blot does not recognise low 14-3-3 protein concentrations. The results showed, that the 14-3-3 protein capture assay in contrast is sensitive enough to detect those 14-3-3 protein concentrations in the liquor, which are not able to be detected by means of the currently used immunoblotting methods.

With a Cut-off of >0.20 OD_{405 nm}, the specificity of the 14-3-3 protein capture assay (calculated by Receiver Operating Characteristic [ROC] curve analysis) was 91.7%. The sensitivity of the assay was 95.1%. (Data obtained from 41 neuropathologically confirmed CJD cases and 36 patients with other neurological diseases; 14-3-3 protein capture assay with ABTS substrate).

Plotting the sensitivity as a function of (100—specificity) for different Cut-off values (ROC-curve) showed a high precision of the test (FIG. 17: exemplary ROC-curve of the 14-3-3 protein capture assay).

The 14-3-3 protein capture assay is also helpful in the pre-symptomatic diagnosis of the Creutzfeldt-Jakob disease and for progression monitoring in case of iatrogenic infection. The little alterations of the 14-3-3 level in iatrogenic Creutzfeldt-Jakob disease are not detectable by conventional immunoblotting methods. Moreover it is possible to employ the 14-3-3 protein capture kit for the novel variant of the Creutzfeldt-Jakob disease (vCJD) and for the progression monitoring of vCJD.

Western Blot analyses of the 14-3-3 protein concentrations in vCJD have hitherto shown lower 14-3-3 protein concentrations, which in comparison to the sporadic Creutzfeldt-Jakob cases are not or only very hardly detectable by this method. The 14-3-3 protein capture assay we have developed for reason of its high sensitivity is very helpful for the diagnosis in vCJD patients.

A further option is the employment of the 14-3-3 protein capture kit in the progression monitoring in the medical therapy of the Creutzfeldt-Jakob disease. Nerve cells, which have been treated with PrP^{Sc} are in vitro submitted to an apoptotic process (Müller et al., Eur. J. Pharmacol. 246: 261-267, 1993). Also a fragment of the prion protein, the peptide PrP106-126, has a neurotoxic effect. There exist know substances having a protective effect on PrP^{Sc} exposed neurons [e.g. Flupirtin (Perovic et al., Neurodegeneration 4: 369-374, 1995; patent DE 195 41 405.5) and Memantin (Müller et al, Eur. J. Pharmacol. 246: 261-267, 1993; patent DE 42 29 805)]. Flupirtin (trade name: Katadolon) is a clinically used, centrally acting non-opiate analgesic. Flupirtin has in vitro a cytoprotective effect counteracting the neurotoxic peptide PrP106-126 (Perovic et al., Neurodegeneration 4: 369-374, 1995). Flupirtin also reduces the extend of apoptosis caused by PrP106-126. The 14-3-3 capture assay thus also allows for a progression monitoring of a medicinal therapy of the Creutzfeldt-Jacob disease, which may possibly become available in the future.

The 14-3-3 protein concentration in bovine liquor is hardly to be determined by means of the conventional Western Blot method. FIG. 18 shows the concentration of 14-3-3γ protein in the liquor from BSE cows (“field cases”), namely determined by A.) 14-3-3 protein capture assay and B.) Western Blot for comparative reasons. A comparison of the results shows, that (1) the concentrations of 14-3-3γ protein in the capture assay are parallel to the band intensities in the Western Blot, and (2) that the increased concentrations of 14-3-3γ protein—in contrast to the 14-3-3 protein capture assay—are hardly detectable in the Western Blot (FIG. 18: exemplary experiment for the representation of the concentrations of 14-3-3γ protein in liquor samples from BSE cows (“field cases”). Compare 14-3-3 protein capture assay (A) and Western Blot (B)).

The 14-3-3 protein capture assay also allows for a determination of the 14-3-3γ protein concentration in the serum of BSE cows (FIG. 19: Chart for illustrating the concentration of 14-3-3γ protein in serum samples from “field cases” of BSE cows).

Also possible by means of the 14-3-3 protein capture assay is a determination of the 14-3-3γ protein concentration in plasma samples from BSE cows (FIG. 20: Concentration of 14-3-3γ protein in plasma samples from BSE cows and control cows).

The 14-3-3 protein capture assay is also suitable to determine the increase of the 14-3-3 protein concentration in the liquor of cows, which were experimentally infected by feeding BSE brain material (FIG. 21: Increase of the 14-3-3 protein content in the liquor of cows which were infected by feeding BSE brain material).

Methods to Prevent Cell-Lysis During the Serum/Plasma Preparation

Comparative experiments with EDTA blood, the platelets of which were separated by centrifugation, and serum in the developed method (14-3-3 protein capture assay) showed that—for reason of cell lysis during the preparation of the serum samples—EDTA blood after the removal of platelets showed better results than the serum.

Therefore, EDTA-blood (the platelets of which were removed) was collected instead of serum, thereby using the following protocol:

1. Withdrawal of EDTA blood (10 ml)

2. Centrifugation at 300×g, 10 min, room temperature

3. Discarding the pellet

4. Centrifugation of the supernatant at 1500×g for 30 min at room temperature

5. Discarding the pellet and freezing of 1 ml aliquots of the supernatant in cryotubes.

The collection and isolation of platelets, lymphocytes and plasma samples from undiluted blood of BSE cows was accomplished according to the following scheme:

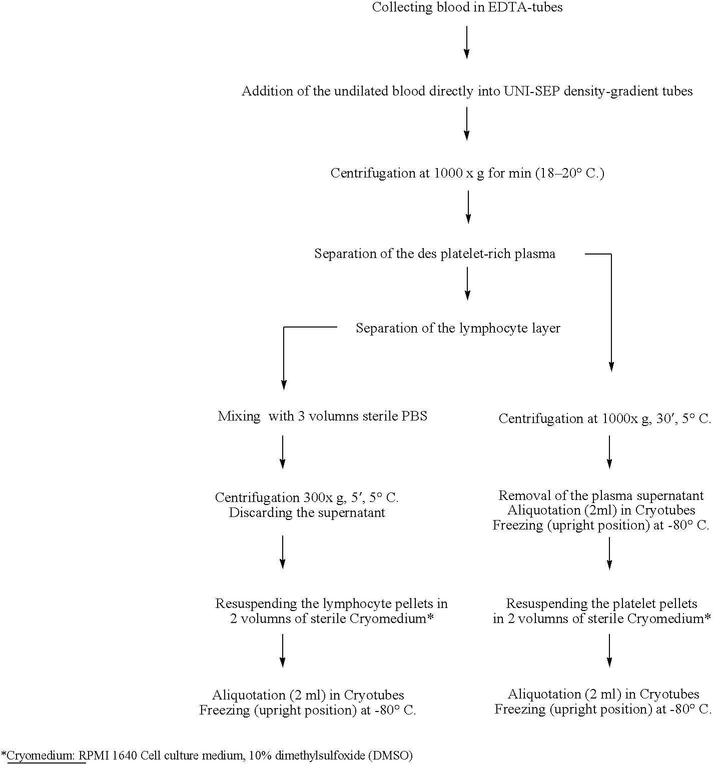

Occurrence of 14-3-3 Protein in Blood Components

The 14-3-3 protein capture assay also allows to determine the 14-3-3 protein in corpuscular blood components.

The occurrence of 14-3-3 protein in platelets, lymphocytes and. plasma was determined by means of the 14-3-3 protein capture assay and the Western Blot (FIG. 22). The lymphocytes were obtained by centrifugation in Uni-Sep tubes. The platelets were isolated from the supernatant.

In the 14-3-3 protein capture assay, the highest 14-3-3γ protein concentration was detected in lymphocyte extracts (about 70 μg/ml; FIG. 22). The 14-3-3γ protein concentration in platelets was 23 μg/ml. The 14-3-3γ protein concentrations of the cell-free plasma and the lymphocyte-free plasma were 37 μg/ml and 26 μg/ml, respectively. A 14-3-3γ protein concentration, which is higher in lymphocytes than in plasma, was also detected by means of the Western Blot (FIG. 23).

Comparison of Different Methods for the Preparation of Plasma/Serum.

Lymphocytes and platelets contain significant amounts of 14-3-3γ protein. Therefore, different methods for the preparation of plasma/serum were compared in order to find that procedure, which will avoid a leakage of 14-3-3γ protein from cellular blood components. The concentration of 14-3-3γ protein was determined in plasma/serum, which was prepared according to the procedures depicted in FIG. 24.

Plasma 1 is obtained by means of Uni-Sep tubes after the removal of lymphocytes. Plasma 2 is lymphocyte- and platelet-free plasma. Plasma 3 is obtained after the separation of lymphocytes and platelets. Plasma 4 is prepared from EDTA-blood by coagulation following the addition of calcium. The serum samples are normal serum (serum 5) and cell-free serum (serum 6). PBS pH 7.4 was used as a negative control. Plasma 2 and plasma 3 showed the lowest OD-values in the 14-3-3 protein capture assay (FIG. 25).

Table 4 summarizes the characteristics of the 14-3-3 protein capture assay (being based on the analysis of 41 neuropathologically confirmed CJD cases and 36 patients with other neurological diseases). The Cut-off value was >0,20 OD405nm, the specificity (=number of negative results divided by the total of negative and false positive results) 91.7% and the sensitivity (=number of positive results divided by the total of positive and false negative results) 95.1%.

In order to determine the detection limit of 14-3-3 protein in the 14-3-3 protein capture assay and in the immunoblot, a dilution series of bovine brain extract with a known concentration of 14-3-3 protein was prepared and 14-3-3 protein measured therein by means of the two methods. The detection limit of the 14-3-3 protein capture assay was 0.02 ng/ml of 14-3-3 protein. The detection limit of the conventional Western Blot method by comparison is about 4 ng/ml of 14-3-3γ protein; this method is thus much less sensitive than the 14-3-3 protein capture assay.

The inter-assay and intra-assay variation of the 14-3-3 protein capture assay is <5%.

Further advantages of the 14-3-3 protein capture assay are:

no requirement to concentrate the samples

can also be used for the detection of weak alterations of the 14-3-3 concentration as they may manifest at an early stage of the disease, before clinical symptoms become apparent

TABLE 4
Characteristics of the 14-3-3 protein capture assay

| | |
|---|---|
| Neuropathologically confirmed CJD cases: | 41 |
| Patients with other neurological diseases: | 36 |
| Cut-off: | >0.20 OD_{405 nm} |
| Specificity: | 91.7% |
| Sensitivity: | 95.1% |
| (calculated by means of Receiver-Operating- | |
| Characteristic [ROC]-curve analysis) | |
| Detection limit (low values): | |
| 14-3-3-Protein-Capture-Assay: | 0.02 ng/ml 14-3-3-γ-Protein |
| Western-Blot: | 4 ng/ml 14-3-3-γ-Protein |
| Inter-Assay und Intra-Assay- | <5% |
| Variations (14-3-3-Protein-Capture-Assay): | |

1. Method for the detection, determination and/or the quantification of an isoform and/or the entirety of the 14-3-3 protein isoforms from the 14-3-3 protein family in the living or dead organism in the human and in the veterinary field, characterised in that in a biological sample at least one isoform and/or the entirety of the 14-3-3 protein isoforms from the 14-3-3 protein family is/are contacted with synthetic or natural peptides comprising amino acid sequence motifs of the type X(n)-XSXXSXXSX-X(n), in particular CX(n)-XSXXSXXSX-X(n) (SEQ ID NO: 13), wherein X is a variable amino acid and S is serine or phosphoserine, and/or RSXpSXP (SEQ ID NO: 12) and/or a 14-3-3 specific antibody, and that the 14-3-3 protein isoform and/or the entirety of the 14-3-3 protein isoforms is detected, determined and/or quantified by means of affinity binding.

2. Method according to claim 1, characterised in that for the determination, for specific binding or concentration of the 14-3-3 proteins a solid phase is used, which is coated with the synthetic or natural peptides or antibodies being directed against purified or recombinant 14-3-3 proteins or 14-3-3 protein isoforms or peptides derived thereof.

3. Method according to claim 2, characterised in that a microtiter plate is used as the solid phase.

4. Method according to claim 2 or 3, whereat the synthetic or natural peptides used for the specific binding or concentration of the 14-3-3 proteins are coupled by means of the maleimide activated microtiter plates by the reaction of the sulfhydryl group of the N-terminal cysteine.

5. Method according to claim 2 or 3, whereat a streptavidin coated microtiter plate or solid phase is used, to which a biotinylated peptide with the above indicated binding motif of the 14-3-3 protein is coupled, and whereat, for the generation of the peptide-14,3,3 protein complexes, the incubation of a biotinylated peptide with the sample is performed either before or after the peptide has bound to the plate.

6. Method according to claim 2 or 3, whereat a carbodiimide or epoxide activated microtiter plate or solid phase is employed, to which a peptide with the above indicated binding motif(s) of the 14-3-3 protein binds, and whereat, for the generation of the peptide-14,3,3 protein complexes, the incubation of a peptide with the sample is performed either before or after the peptide has bound to the plate.

7. Method according to one of claims 1 to 6, characterised in that the determination of 14-3-3 protein is performed at a solid phase and a mobile phase, whereat the binding peptide and/or the antibody is coupled to the solid phase and the 14-3-3 protein to be determined is present in the mobile phase.

8. Method according to one of claims 2 to 7 in the form of a sandwich assay, characterised in that at least one isoform of the 14-3-3 protein family and/or its entirety is isolated by means of one or more specific capture antibodies being covalently or otherwise bound to solid phases, in particular to microtiter plates, followed by an immediate or delayed determination or quantification by means of a second, specific detection antibody.

9. Method according to one of claims 2 to 8, characterised in that the determination is accomplished in the form of a competition assay.

10. Method according to claim 9, wherein the competition takes place between the 14-3-3 protein to be determined in the sample and purified or recombinant 14-3-3 proteins or 14-3-3 protein isoforms or peptide fragments thereof, all of them competing for the same binding site at the binding peptide or antibody coupled to the solid phase, or whereat it is a competition between soluble 14-3-3 protein binding peptides in the mobile phase and the 14-3-3 protein binding peptide coupled to the solid phase, both competing for the 14-3-3 protein to be determined in the sample.

11. Method according to one of the preceding claims, characterised in that the method is combined in the same assay system or in the same kit with the determination of another biomarker (surrogate marker) or of a pathogenic agent in the same sample mixture by its binding to the same or to another solid phase in the form of a combination assay.

12. Method according to one of the preceding claims in the form of a capture assay, comprising the combination of binding the 14-3-3 protein to be determined to (a) a peptide recognition sequence and (b) to an antibody.

13. Method according to one of the preceding claims, characterised in that the determination of 14-3-3 protein is combined with the determination of a quality marker for the sample to be investigated.

14. Method according to one of the preceding claims, characterised in that the biological sample comprises cells, cell associations, tissues, organic fluid or bodily fluid like blood, serum, plasma, liquor, lacrimal secretion, milk secretion or urine.

15. Method according to one of the preceding claims, whereat the detection, the determination and/or the quantification of the generated peptide-14-3-3 protein complexes is accomplished by means of radionucleotide-, dye- or enzyme-labeled antibodies.

16. Use of the method according to one of the preceding claims for the determination of 14-3-3 binding proteins or 14-3-3 specific antibodies, whereat one measures the decrease of the binding of a pre-selected amount of 14-3-3 protein to the solid phase in the presence of these 14-3-3 binding proteins or 14-3-3 specific antibodies.

17. Use of the method according to one of the preceding claims for the detection and quantification of the 14-3-3 proteins or their isoforms in the diagnosis of TSE-diseases like Creutzfeldt-Jakob disease (CJD) and its novel form in young persons (vCJD), Gerstmann-Sträussler-Scheinker syndrome (GSS), Fatal familial insomnia (FFI), Kuru, Scrapie (Traberkrankheit; Gnubberkrankheit; tremblente de mouton), Bovine Spongiform Encephalopathy (BSE), Transmissible mink encephalopathy (TME), Chronic Wasting Disease of cervine animals (CWD), Spongiform encephalopathies in wild ruminants and Feline spongiform encephalopathy (FSE) in living and/or dead organisms and also of other diseases, which are associated with an alteration of the 14-3-3 protein concentration, and also for progression monitoring of therapies against diseases being associated with the 14-3-3 protein concentration.

18. Use of the method according to one of the preceding claims for the early stage diagnosis of BSE or Creutzfeldt-Jakob disease of the old or novel variant, in liquor, blood serum, plasma or other bodily fluids at the living or dead patient or organism.

19. Use of the 14-3-3 protein family or at least one isoform from the 14-3-3 protein family as a biomarker for determining the effects of xenobiotics of all kind or of natural environmental poisons in aquatic invertebrates and other organisms including the human.
